(19) United States
(12) Patent Application Publication
(10) Pub. No.: US 2026/0007392 A1
(43) Pub. Date: Jan. 8, 2026

(54) METHOD FOR CARDIAC MYOCARDIAL STRAIN AND ULTRASOUND IMAGING APPARATUS

(71) Applicant: Shenzhen Mindray Bio-Medical Electronics Co., Ltd., Shenzhen (CN)

(72) Inventors: Yanbo LIU, Shenzhen (CN); Xing AN, Shenzhen (CN); Longfei CONG, Shenzhen (CN)

(21) Appl. No.: 19/263,180

(22) Filed: Jul. 8, 2025

(30) Foreign Application Priority Data
Jul. 8, 2024 (CN) 202410911740.4

(51) Int. Cl. A61B 8/00; A61B 8/08; G06T 7/00; G06T 7/246; G06T 11/20

(52) U.S. Cl. CPC A61B 8/485; A61B 8/0883; A61B 8/463; A61B 8/5207; G06T 7/0012; G06T 7/246; G06T 11/206; G06T 2207/10132; G06T 2207/30048; G06T 2210/41

(57) ABSTRACT

Disclosed are a method for presenting cardiac myocardial strain and an ultrasound imaging apparatus, comprising: acquiring image data of myocardial segments of both left and right ventricles, and processing this data to obtain motion parameters of both left and right ventricular myocardial segments. A composite bull's-eye plot containing left and right ventricular bull's-eye subplots is displayed, wherein the left ventricular bull's-eye subplot comprises multiple left ventricular myocardial regions corresponding to the left ventricular myocardial segments and presenting motion parameters of the left ventricular myocardial segment; and the right ventricular bull's-eye subplot comprises a right ventricular myocardial region corresponding to the right ventricular myocardial segments that exclude some/all shared segments with the left ventricle and presenting motion parameters of the right ventricular myocardial segment. This configuration enables users to observe motion parameters of both ventricular myocardial segments, thereby providing comprehensive understanding of cardiac myocardial strain while enhancing clinical efficiency for doctors.

### CROSS-REFERENCE TO RELATED APPLICATIONS

This application claims priority to Chinese Patent Application No. 202410911740.4, entitled “METHOD FOR CARDIAC MYOCARDIAL STRAIN AND ULTRASOUND IMAGING APPARATUS” filed on Jul. 8, 2024 the content of which is incorporated herein by reference in its entirety.

### TECHNICAL FIELD

The present disclosure relates to the field of medical devices and, more particularly, to methods for presenting cardiac myocardial strain and ultrasound imaging apparatus.

### BACKGROUND

Ultrasound is an interdisciplinary field integrating acoustics, optics, electronics, and medicine. Diagnostic ultrasound imaging employs ultrasound waves to produce real-time blood flow imaging along with two-dimensional (2D) and three-dimensional (3D) anatomical medical imaging. Ultrasound refers to sound waves with frequencies higher than the audible range for humans. The frequency range used in echocardiography spans from 2 MHz (for adult transthoracic scanning) to 7 MHz (for harmonic imaging, pediatric examinations, or transesophageal ultrasound).

Echocardiography is imaged via pulsed-echo technology. The probe generates ultrasound pulses of 2-3 cycles directed toward a patient. These pulses produce echoes at organ boundaries or within internal tissues. The echoes are detected by the probe and transmitted to the display of the ultrasound equipment. The equipment processes the echo signals and presents them as speckles, forming anatomical images visible on the screen. The brightness of the speckles corresponds to the echo intensity, and the position of each speckle aligns with the anatomical location of the echo-generating structure. Positional information is determined by the direction of the pulse wave and the time taken for the echo to return to the probe. The top of the ultrasound image represents structures closest to the probe. Assuming a constant speed of sound propagation, the depth of the echo-generating structure is calculated based on the echo return time. Driven by advancements in image processing and clinical utility, quantitative analysis methods to evaluate myocardial tissue motion—thereby assessing cardiac contractility—have become one of the most promising and high-value research directions in echocardiography.

Speckle Tracking Technology is widely utilized to trace acoustic speckles formed by ultrasound echoes, identifying motion trajectories of stationary speckles to aid users in understanding myocardial movement in patients. Additionally, within any imaging plane and cardiac cycle, this technique analyzes myocardial deformation and displacement across multiple directions, enabling comprehensive assessment of the left ventricle. The longitudinal strain (LS) proposed by this technique has emerged as one of the most clinically promising cardiac function evaluation metrics following ejection fraction (EF). Global longitudinal strain (GLS), defined as the aggregate longitudinal strain score, represents the average of peak longitudinal strains across all myocardial segments. Beyond this parameter, two-dimensional speckle tracking also facilitates the acquisition of motion velocity, volumetric data, and radial strain. In recent years, the Chinese Medical Doctor Association, European Association of Cardiovascular Imaging, and American Society of Echocardiography have endorsed normal reference values for LS based on 2D speckle tracking echocardiography (2D STE) in their respective guidelines and consensus documents, further promoting the standardization and widespread adoption of strain analysis technologies.

Within strain analysis functionalities, the “bull's-eye plot” serves as a critical visualization modality, schematically illustrated in FIG. 1. Currently, three models are clinically prevalent: 16-, 17-, and 18-segment configurations. Taking the 18-segment model as an example (see the rightmost bull's-eye plot in FIG. 1), the disk is divided into three columns, each corresponding to myocardial segments from three cardiac imaging planes: A\2\3\4C.

However, existing strain bull's-eye plots still exhibit certain shortcomings. Historically, doctors have predominantly focused on quantitative analysis of the left ventricle (LV), primarily for evaluating LV systolic function. Consequently, current bull's-eye plots primarily display strain values of LV myocardial segments across cardiac phases, demonstrating inherent limitations that impede comprehensive assessment of myocardial strain for the entire heart.

Therefore, the existing presentation methods of myocardial strain still need improvement and enhancement.

### SUMMARY

The present disclosure primarily provides methods for presenting cardiac myocardial strain and ultrasound imaging apparatus, intended to provide doctors with more myocardial strain information and improve clinical efficiency.

A method for presenting cardiac myocardial strain is provided in some embodiments, wherein a left ventricle of a heart comprises a plurality of myocardial segments and a right ventricle of the heart comprises a plurality of myocardial segments, several of the myocardial segments are shared by both the left ventricle and the right ventricle; the method comprising:
acquiring first ultrasound image data of a cardiac imaging plane, the first ultrasound image data containing image data of the myocardial segments of the left ventricle and image data of the myocardial segments of the right ventricle;
processing the first ultrasound image data to obtain a motion parameter of the myocardial segments of the left ventricle and a motion parameter of the myocardial segments of the right ventricle;
displaying a composite bull's-eye plot of the left ventricle and the right ventricle, the composite bull's-eye plot comprising a left ventricular bull's-eye subplot and a right ventricular bull's-eye subplot; wherein:
(1) the left ventricular bull's-eye subplot comprises a plurality of left ventricular myocardial regions corresponding to the plurality of myocardial segments of the left ventricle, the motion parameter of the myocardial segments of the left ventricle is presented in its corresponding left ventricular myocardial regions; and,
the right ventricular bull's-eye subplot comprises at least one right ventricular myocardial region corresponding to the myocardial segments of the right ventricle excluding some or all of the shared myocardial segments, the motion parameter of said myocardial segments of the right ventricle is presented in its corresponding right ventricular myocardial region(s); or
(2) the left ventricular bull's-eye subplot comprises at least one left ventricular myocardial region corresponding to the myocardial segments of the left ventricle excluding some or all of the shared myocardial segments, the motion parameter of said myocardial segments of the left ventricle is presented in its corresponding left ventricular myocardial region(s); and, the right ventricular bull's-eye subplot comprises a plurality of right ventricular myocardial regions corresponding to the plurality of myocardial segments of the right ventricle, the motion parameter of the myocardial segments of the right ventricle is presented in its corresponding right ventricular myocardial regions.

A positional relationship between the left ventricular bull's-eye subplot and the right ventricular bull's-eye subplot in the composite bull's-eye plot is consistent with a positional relationship between the left ventricle and the right ventricle on a short-axis section; and, the composite bull's-eye plot further comprises an interventricular septum region configured to present an interventricular septum, the interventricular septum region is adjacent to the left ventricular bull's-eye subplot and/or the right ventricular bull's-eye subplot.

The right ventricular myocardial regions in the right ventricular bull's-eye subplot are sequentially arranged vertically along a curve; the right ventricular bull's-eye subplot, the interventricular septum region, and the left ventricular bull's-eye subplot are arranged sequentially from left to right or from right to left; or, the right ventricular bull's-eye subplot and the interventricular septum region are arranged vertically one above the other and both are adjacent to the left ventricular bull's-eye subplot.

A positional relationship between the left ventricular bull's-eye subplot and the right ventricular bull's-eye subplot in the composite bull's-eye plot is consistent with a positional relationship between the left ventricle and the right ventricle on a short-axis section; and the left ventricular bull's-eye subplot is adjacent to the right ventricular bull's-eye subplot.

The right ventricular myocardial regions in the right ventricular bull's-eye subplot are sequentially arranged vertically along a curve; or, the right ventricular myocardial regions in the right ventricular bull's-eye subplot are arranged sequentially in a left-right orientation.

The right ventricular myocardial regions in the right ventricular bull's-eye subplot comprise one or more of: a myocardial region corresponding to a top segment of a right ventricular free wall, a myocardial region corresponding to a mid segment of the right ventricular free wall, and a myocardial region corresponding to a bottom segment of the right ventricular free wall; and, the left ventricular myocardial regions in the left ventricular bull's-eye subplot comprise: 16 myocardial regions corresponding respectively to 16 myocardial segments of the left ventricle, 17 myocardial regions corresponding respectively to 17 myocardial segments of the left ventricle, or 18 myocardial regions corresponding respectively to 18 myocardial segments of the left ventricle.

Acquiring second ultrasound image data of a/the cardiac imaging plane, the second ultrasound image data containing image data of a left atrium of the heart and image data of a right atrium of the heart; processing the second ultrasound image data to obtain a motion parameter of the left atrium and a motion parameter of the right atrium; and, displaying an atrial model diagram, the atrial model diagram being configured to display the motion parameter of the left atrium and the motion parameter of the right atrium.

The composite bull's-eye plot and the atrial model diagram are displayed in a same display interface; or, displaying an atrial model diagram comprises: receiving a first switching instruction; and switching from displaying the composite bull's-eye plot to displaying the atrial model diagram in response to the first switching instruction.

The atrial model diagram comprises: a left atrial region corresponding to the left atrium, a right atrial region corresponding to the right atrium, and an aortic region corresponding to an aorta of the heart; wherein the left atrial region displays the motion parameter of the left atrium, and the right atrial region displays the motion parameter of the right atrium.

The atrial model diagram further comprises: a right ventricular outflow tract region corresponding to a right ventricular outflow tract of the heart, and a pulmonary artery region corresponding to a pulmonary artery of the heart; and, a positional relationship among the left atrial region, the right atrial region, the aortic region, the right ventricular outflow tract region and the pulmonary artery region in the atrial model diagram are consistent with positions of corresponding left atrium, right atrium, aorta, right ventricular outflow tract and pulmonary artery on a short-axis section.

The atrial model diagram further comprises: a right ventricular outflow tract region corresponding to a right ventricular outflow tract of the heart, and a pulmonary artery region corresponding to a pulmonary artery of the heart; the aortic region is located centrally; and the left atrial region, the right atrial region, the right ventricular outflow tract region and the pulmonary artery region are sequentially arranged circumferentially around the aortic region.

Processing the first ultrasound image data to obtain a motion parameter of the myocardial segments of the left ventricle and a motion parameter of the myocardial segments of the right ventricle, comprises: determining a contour of the left ventricle and a contour of the right ventricle in the first ultrasound image data; and, obtaining the motion parameter of the myocardial segments of the left ventricle and the motion parameter of the myocardial segments of the right ventricle, based on the contour of the left ventricle and the contour of the right ventricle in the first ultrasound image data.

Acquiring third ultrasound image data of a/the cardiac imaging plane, the third ultrasound image data containing image data of the myocardial segments of the left ventricle and image data of the left atrium; processing the third ultrasound image data to obtain the motion parameter of the myocardial segments of the left ventricle and the motion parameter of the left atrium; and, displaying a myocardial segment model diagram, wherein the myocardial segment model diagram comprises: a plurality of left ventricular myocardial regions corresponding to the plurality of myocardial segments of the left ventricle, and a left atrial region corresponding to the left atrium; the motion parameter of the myocardial segments of the left ventricle is presented in its corresponding left ventricular myocardial regions; and the motion parameter of the left atrium is presented in the left atrial region.

The composite bull's-eye plot and the myocardial segment model diagram are displayed in a same display interface; or, displaying the myocardial segment model diagram comprises: receiving a second switching instruction; and switching from displaying the composite bull's-eye plot to displaying the myocardial segment model diagram in response to the second switching instruction; or, displaying the myocardial segment model diagram comprises: receiving a third switching instruction; and switching from displaying the composite bull's-eye plot and the atrial model diagram to displaying the myocardial segment model diagram in response to the third switching instruction.

The myocardial segment model diagram further comprises an aortic region corresponding to the aorta; in the myocardial segment model diagram, the left ventricular myocardial regions are arranged to form a contour of the left ventricle in a cardiac imaging plane; and, a positional relationship among the plurality of left ventricular myocardial regions, the left atrial region and the aortic region in the myocardial segment model diagram are consistent with a positional relationship among their corresponding left ventricle, left atrium, and aorta in a cardiac imaging plane.

The third ultrasound image data further contains image data of the myocardial segments of the right ventricle of the heart, and image data of the right atrium; the third ultrasound image data is processed to obtain the motion parameter of the myocardial segments of the right ventricle and the motion parameter of the right atrium; the myocardial segment model diagram further comprises: a plurality of right ventricular myocardial regions corresponding to the plurality of myocardial segments of the right ventricle, and a right atrial region corresponding to the right atrium; the motion parameter of the myocardial segments of the right ventricle is presented in its corresponding right ventricular myocardial regions; and, the motion parameter of the right atrium is presented in the right atrial region.

In the myocardial segment model diagram, the left ventricular myocardial regions are arranged to form a contour of the left ventricle in a cardiac imaging plane; the right ventricular myocardial regions are arranged to form a contour of the right ventricle in a cardiac imaging plane; a positional relationship among the plurality of left ventricular myocardial regions, the left atrial region, the plurality of right ventricular myocardial regions, and the right atrial region in the myocardial segment model diagram are consistent with positions of corresponding left ventricle, left atrium, right ventricle, and right atrium on a cardiac imaging plane.

A method for presenting cardiac myocardial strain provided in some embodiments comprises:
acquiring second ultrasound image data of a cardiac imaging plane, the second ultrasound image data containing image data of a left atrium of a heart and image data of a right atrium of the heart;
processing the second ultrasound image data to obtain a motion parameter of the left atrium and a motion parameter of the right atrium; and
displaying an atrial model diagram, the atrial model diagram being configured to display the motion parameter of the left atrium and the motion parameter of the right atrium, wherein the atrial model diagram comprises: a left atrial region corresponding to the left atrium, a right atrial region corresponding to the right atrium, and an aortic region corresponding to an aorta of the heart; the left atrial region displays the motion parameter of the left atrium; and the right atrial region displays the motion parameter of the right atrium.

A method for presenting cardiac myocardial strain is provided in some embodiments, wherein the left ventricle of a heart comprises a plurality of myocardial segments; and the method comprises:
acquiring third ultrasound image data of a cardiac imaging plane, the third ultrasound image data containing image data of at least one of the myocardial segments of the left ventricle and image data of a left atrium of the heart;
processing the third ultrasound image data to obtain a motion parameter of the at least one of the myocardial segments of the left ventricle and a motion parameter of the left atrium; and
displaying a myocardial segment model diagram; wherein the myocardial segment model diagram comprises: a plurality of left ventricular myocardial regions corresponding to the plurality of myocardial segments of the left ventricle and a left atrial region corresponding to the left atrium, the motion parameter of the at least one of the myocardial segments of the left ventricle is presented in its corresponding left ventricular myocardial region(s), and the motion parameter of the left atrium is presented in the left atrial region.

The myocardial segment model diagram further comprises an aortic region corresponding to an aorta of the heart; in the myocardial segment model diagram, the left ventricular myocardial regions are arranged to form a contour of the left ventricle on a cardiac imaging plane; a positional relationship among the plurality of left ventricular myocardial regions, the left atrial region, and the aortic region in the myocardial segment model diagram are consistent with a positional relationship among corresponding left ventricle, left atrium, and aorta on a cardiac imaging plane.

The third ultrasound image data further contains image data of a plurality of myocardial segments of a right ventricle of the heart and image data of a right atrium of the heart; the third ultrasound image data is processed to obtain a motion parameter of at least one of the myocardial segments of the right ventricle and a motion parameter of the right atrium; the myocardial segment model diagram further comprises: a plurality of right ventricular myocardial regions corresponding to the plurality of myocardial segments of the right ventricle and a right atrial region corresponding to the right atrium, the motion parameter of the at least one of the myocardial segments of the right ventricle is presented in its corresponding right ventricular myocardial region(s), and the motion parameter of the right atrium is presented in the right atrial region.

In the myocardial segment model diagram, the left ventricular myocardial regions are arranged to form a contour of the left ventricle on a cardiac imaging plane, and the right ventricular myocardial regions are arranged to form a contour of the right ventricle on said cardiac imaging plane; a positional relationship among the plurality of left ventricular myocardial regions, the left atrial region, the plurality of right ventricular myocardial regions, and the right atrial region in the myocardial segment model diagram are consistent with a positional relationship among corresponding left ventricle, left atrium, right ventricle, and right atrium on said cardiac imaging plane.

And the motion parameter comprises a strain, a strain rate, a velocity, a displacement, or a contrast agent time-to-peak.

An ultrasound imaging apparatus provided in some embodiments comprises:
an ultrasound probe;
a transmitting circuit configured to excite the ultrasound probe to transmit ultrasound waves;
a receiving circuit configured to control the ultrasound probe to receive echoes of the ultrasound waves;
a human-machine interaction device configured to output visual information and receive user input;
a memory configured to store a program; and
a processor configured to execute the program to implement the method mentioned above.

A computer-readable storage medium is provided in some embodiments, wherein the medium stores a program executable by a processor to implement the method mentioned above.

According to the method for presenting cardiac myocardial strain and the ultrasound imaging apparatus in the above-mentioned embodiments, image data of at least one myocardial segment of the left and right ventricles of a heart is obtained by acquiring ultrasound image data of a cardiac imaging plane, and motion parameters of at least one myocardial segment of the left and right ventricles can further be obtained by processing these image data. A composite bull's-eye plot containing a left ventricular bull's-eye subplot and a right ventricular bull's-eye subplot can be displayed, wherein: the left ventricular bull's-eye subplot comprises a plurality of left ventricular myocardial regions of the plurality of myocardial segments of the left ventricle, with the motion parameter of the left ventricular myocardial segment being presented in its corresponding left ventricular myocardial region; and the right ventricular bull's-eye subplot comprises a right ventricular myocardial region corresponding to myocardial segments of the right ventricle excluding some or all of the myocardial segments shared with the left ventricle, with the motion parameter of the right ventricular myocardial segment being presented in its corresponding right ventricular myocardial region. This configuration allows users to visualize motion parameters of the left and right ventricular myocardium, thereby enabling a comprehensive understanding of cardiac myocardial strain conditions and improving doctors' clinical workflow efficiency.

### BRIEF DESCRIPTION OF THE DRAWINGS

FIG. 1 is left ventricular bull's-eye plots illustrating conventional 16-, 17-, and 18-myocardial segments;

FIG. 2 is a structural block diagram of a cardiac ultrasound auxiliary analysis system provided in some embodiments of the present disclosure;

FIG. 3 is a flowchart of a method for presenting cardiac myocardial strain provided in some embodiments of the present disclosure;

FIG. 4 is a schematic diagram showing relative positional relationship between multiple cardiac imaging planes and a heart;

FIG. 5 is a flowchart of step 2 shown in FIG. 3 in some embodiments;

FIG. 6 is a schematic diagram illustrating left ventricular contour marked by multiple points in an ultrasound image;

FIG. 7 is a schematic diagram illustrating key point tracking;

FIGS. 8-13 are schematic diagrams of composite bull's-eye plots in various embodiments;

FIG. 14 is a schematic diagram illustrating 16-, 17-, and 18-myocardial segments of the left ventricle, respectively;

FIG. 15 is a schematic diagram of left ventricular myocardial segments contained in ultrasound images in three cardiac imaging planes;

FIG. 16 is a schematic diagram showing left and right ventricular myocardial segments contained in an ultrasound image in the A4C imaging plane;

FIG. 17 is a flowchart of a method for presenting cardiac myocardial strain provided in some embodiments of the present disclosure;

FIG. 18 is a schematic diagram of an atrial model diagram in some embodiments;

FIG. 19 is a flowchart of a method for presenting cardiac myocardial strain provided in some embodiments of the present disclosure;

FIGS. 20-22 are schematic diagrams of myocardial segment model diagrams in different embodiments; and

FIG. 23 is a structural block diagram of an ultrasound imaging apparatus provided in some embodiments of the present disclosure.

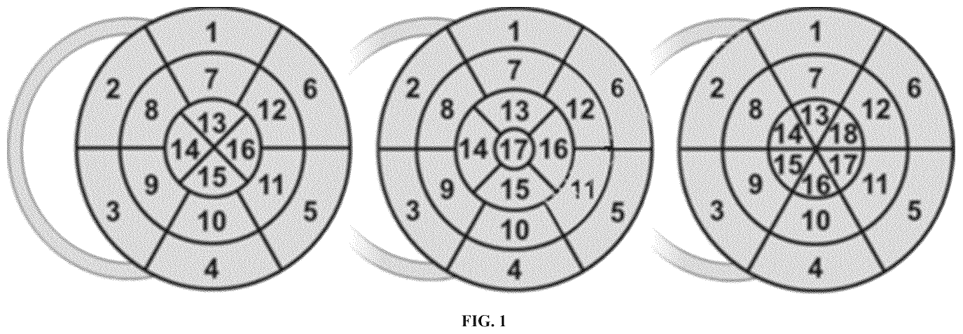

### DESCRIPTION OF THE EMBODIMENTS

Specific embodiments of the present disclosure will now be described in detail with reference to the accompanying drawings. Similar or related components in different embodiments are labeled with associated reference numerals. The following embodiments include detailed descriptions to facilitate understanding of the present disclosure. However, those skilled in the art will readily recognize that certain features may be omitted under specific circumstances or substituted by other components, materials, or methods. In some instances, certain operations related to the present disclosure are not explicitly described or illustrated herein. This intentional exclusion is intentional to avoid obscuring the core technical solutions of the present disclosure. For those skilled in the art, a complete understanding of these operations can be attained through the descriptions provided in this specification and general technical knowledge in the art.

Additionally, the features, operations, or characteristics described in the specification may be combined in any suitable manner to form various embodiments. Similarly, steps or actions in the method descriptions may be reordered or modified in ways that would be obvious to those skilled in the art. Therefore, the sequences presented in the specification and drawings are intended solely to clarify the description of specific embodiments and do not imply mandatory orderings, unless explicitly stated that a particular sequence is required.

The numerical designations assigned to components in this specification, such as ‘first,’ ‘second,’ or similar ordinal terms, serve solely to distinguish described objects and carry no inherent sequential or technical implications. Furthermore, the terms ‘connected’ and ‘coupled’ as used herein encompass both direct and indirect connection (coupling), unless explicitly stated otherwise.

According to the present disclosure, ultrasound image data of multiple cardiac chambers is processed to obtain myocardial motion parameters of the multiple chambers, which is capable of characterizing myocardial strain, and provides a composite display of the multi-chamber myocardial motion parameters, thereby optimizing diagnostic workflows, enhancing clinical efficiency, and expanding diagnostic information dimensions for doctors. Detailed descriptions of exemplary embodiments are provided below.

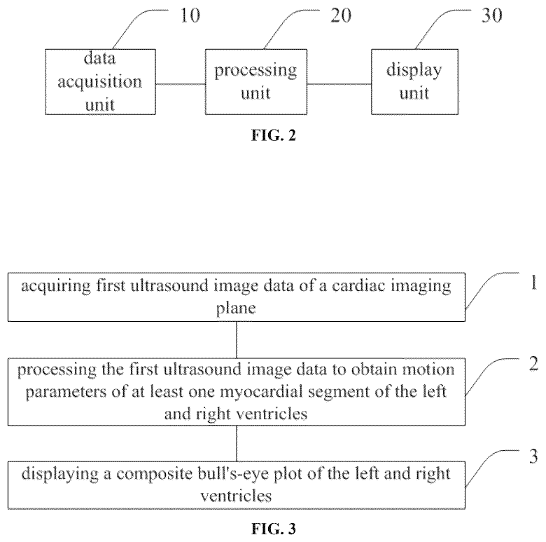

As shown in FIG. 2, a cardiac ultrasound auxiliary analysis system provided in some embodiments of the present disclosure may comprise: a data acquisition unit 10, a processing unit 20, and a display unit 30.

The data acquisition unit 10 is configured to acquire ultrasound image data of any cardiac imaging plane. The ultrasound image data may comprise cine data of any cardiac imaging plane suitable for strain analysis. The cardiac imaging planes are not limited by cardiac chambers and may include imaging planes of the left ventricle, left atrium, right ventricle, right atrium, etc. The cardiac ultrasound examination is not restricted by scanning mode and may include pediatric, adult, neonatal, or fetal cardiac examinations. The cardiac imaging planes are not confined to specific view types, which may include parasternal views, apical views, suprasternal notch views, subcostal views, etc. The cardiac imaging planes may encompass both long-axis and short-axis orientations. The ultrasound image data is not limited by modality and may include B-mode images, contrast-enhanced images (obtained in contrast mode), or other formats.

The processing unit 20 is configured to process the ultrasound image data acquired by the data acquisition unit 10 to obtain motion parameters of myocardial segments and/or atrial motion parameters.

The display unit 30 is configured to comprehensively display the motion parameters of myocardial segments and/or atrial motion parameters, enabling doctors to obtain more myocardial strain information and thereby improving workflow efficiency.

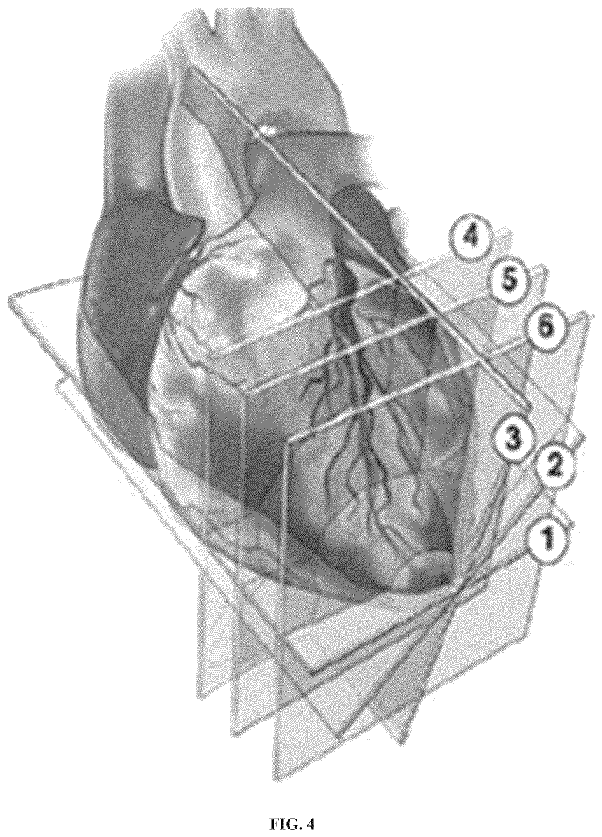

The cardiac ultrasound auxiliary analysis system may employ multiple modalities for presenting cardiac myocardial strain, several of which are described below. One such presentation modality for cardiac myocardial strain is illustrated in FIG. 3 and comprises the following steps:

Step 1: the processing unit 20 acquires first ultrasound image data of a cardiac imaging plane by the data acquisition unit 10. The left ventricle of the heart comprises a plurality of myocardial segments, and the right ventricle of the heart comprises a plurality of myocardial segments. There are several myocardial segments shared by both the myocardial segments of the left ventricle and the myocardial segments of the right ventricle. The division of myocardial segments in the left and right ventricles may be predefined. For example, the plurality of myocardial segments contained in the left ventricle may be conventional 16, 17, or 18 myocardial segments, while the plurality of myocardial segments contained in the right ventricle may include segments such as the apical segment of a right ventricular free wall, the mid segment of the right ventricular free wall, and the basal segment of the right ventricular free wall. In some embodiments, the myocardial segments of the left and right ventricles may be divided according to guidelines established by the Chinese Medical Doctor Association, the European Association of Cardiovascular Imaging, or the American Society of Echocardiography.

The first ultrasound image data primarily comprises ultrasound video data, i.e., sequential ultrasound image frames. Specifically, the first ultrasound image data includes image data of at least one myocardial segment of the left ventricle and image data of at least one myocardial segment of the right ventricle. This indicates that the cardiac imaging plane intersects both left and right ventricles, thereby including images of both the left and right ventricles in the ultrasound image data. The cardiac imaging plane may encompass any imaging plane of the heart, provided it spans both the left and right ventricles in the embodiment of FIG. 3. Typically, this may correspond to standard echocardiographic views, such as one of the six imaging planes illustrated in FIG. 4: A4C view (apical four-chamber view), A2C view (apical two-chamber view), A3C view (apical three-chamber view), basal short-axis view, papillary muscle short-axis view, and apical short-axis view. The first three views are aligned with the long-axis orientation, while the latter three are aligned with the short-axis orientation.

The first ultrasound image data may be acquired by the data acquisition unit 10 through clinical scanning or retrieved from storage media. For example, the data acquisition unit 10 includes a transmitter unit and a receiver unit. The transmitter unit emits ultrasound waves toward the heart via an ultrasound probe, while the receiver unit receives echo signals. The received echo signals undergo signal processing steps such as beamforming to generate the first ultrasound image data. The first ultrasound image data may comprise historical ultrasound video data or real-time ultrasound video data. For example, real-time ultrasound video data may include ultrasound images displayed live on the user interface of an ultrasound imaging apparatus or image sequences reviewed via trackball scrolling after freezing (e.g., using the “Freeze” function). Historical ultrasound video data, also referred to as offline ultrasound video data, may include cine data stored during prior cardiac scans. This embodiment addresses doctors' need to evaluate myocardial strain following ultrasound scanning of cardiac imaging planes. Accordingly, the data acquisition unit 10 is described herein primarily in the context of acquiring real-time first ultrasound image data through live ultrasound scanning of a imaging plane. Additionally, the data acquisition unit 10 may obtain the first ultrasound image data from external devices, such as imported ultrasound video data.

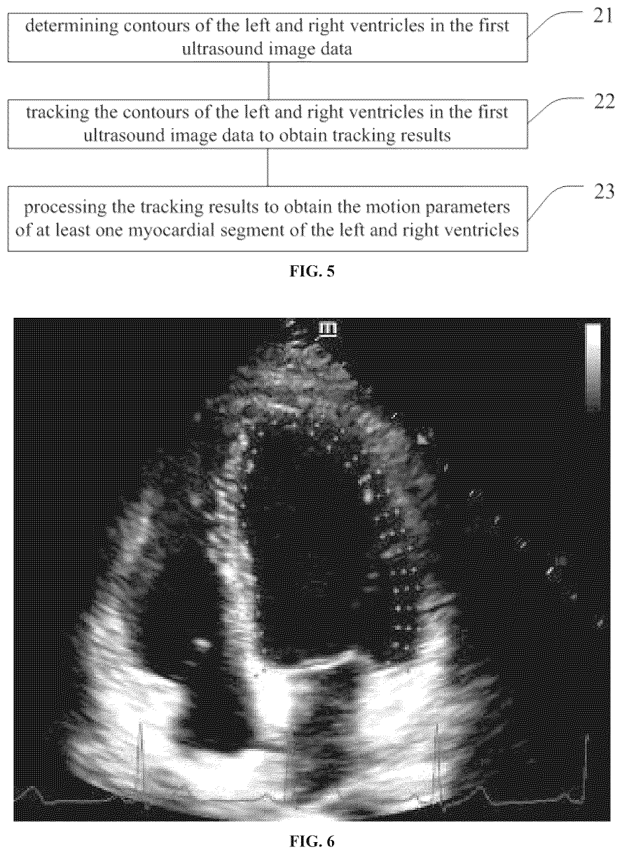

Step 2: the processing unit 20 processes the first ultrasound image data to obtain a motion parameter of at least one myocardial segment of the left ventricle and a motion parameter of at least one myocardial segment of the right ventricle. A detailed workflow is illustrated in FIG. 5 and includes the following steps:

Step 21: the processing unit 20 determines a contour of the left ventricle and a contour of the right ventricle in the first ultrasound image data. This step may be implemented through multiple modalities, three of which are exemplarily described below.

First Approach: The processing unit 20 identifies the positions of the left and right ventricles based on a predefined detection algorithm, then performs segmentation (or extraction) of the left ventricular boundary using a predefined segmentation algorithm to generate the contour of the left ventricle. Similarly, the right ventricular boundary is segmented (or extracted) using the predefined segmentation algorithm to generate the contour of the right ventricle.

The predefined detection algorithm may include, but is not limited to, one or more of the following: deep learning models, machine learning models, or traditional image processing algorithms.

For example, the predefined detection algorithm may comprise a pre-trained deep learning model designed to detect regions of interest (ROIs), specifically the left and right ventricular regions. Prior to this, ultrasound images of cardiac imaging planes may be collected as first training images, with annotations of the left and right ventricular regions created by delineating key anatomical structures such as the endocardium, epicardium, or myocardium by senior clinicians to represent the regions of the left and right ventricles. These annotated training images, along with their corresponding labels (such as bounding box for ROIs, i.e., coordinate information), are used to train the deep learning model. During the model training stage, the error between the detection results of the left and right ventricles and the annotated results is calculated in the iterative process, with the weights in the network continuously updated to minimize prediction errors. This process is repeated continuously until the detection results gradually approach the true values of the left and right ventricular ROIs, obtaining a trained deep learning model (ROI detection model). This model enables automated detection and extraction of the left and right ventricles from newly input ultrasound image data. When the processing unit 20 inputs the first ultrasound image data into this model, the contours of the left and right ventricles in the ultrasound image can be obtained. The detection and segmentation networks used in the deep learning model may include but are not limited to RCNN, FasterRCNN, SSD, YOLO, etc.

For another example, the predefined detection algorithm may incorporate traditional image processing algorithms as well as machine learning models. The processing unit 20 may identify candidate regions based on image processing methods (such as the Select Search algorithm), subsequently resize the candidate regions to a predetermined dimensions, and extract image features including gradients and textures through image processing techniques such as SIFT operators, HOG operators, or GLCM (Gray-Level Co-occurrence Matrix). The feature vectors of the candidate regions are trained using conventional machine learning algorithms to obtain the classification model of the candidate boxes. The bounding box of the target is obtained through regression methods.

Second Approach: The processing unit 20 performing an integrated detection and segmentation of the left and right ventricles based on a preset segmentation algorithm, which may be semi-automatic or fully automatic algorithms. The segmentation algorithms in both the first and second approaches may employ the same segmentation principle. The difference lies in that the first approach performs segmentation on the detected regions (locations) of the left and right ventricles, while the second approach directly segments the ultrasound image. Both approaches ultimately segment the contours of the left and right ventricles.

The segmentation algorithms in these two approaches can be specifically implemented in various ways, as exemplified below.

For instance, the segmentation algorithm may be a deep learning model based on a segmentation network, with primary segmentation networks including Unet, FCN, and their improved variants. In the first approach, the deep learning model can extract the boundaries of the ROI regions detected, obtaining the contours of the left and right ventricles. In the second approach, the deep learning model can extract the boundaries of the ultrasound images in the first ultrasound image data, obtaining the contours of the left and right ventricles. The deep learning model is trained using second training images and their annotated regions. The second training images are ultrasound images of cardiac imaging planes, and the annotated regions may be binary images of the left and right ventricles or annotation files formed by writing the position information of the left and right ventricles into XML or JSON. By inputting the second training images into this deep learning model, the error between the segmentation results output by the model and the annotated results is continuously iterated to minimize the error until the segmentation results approach the true values, thereby obtaining a well-trained deep learning model.

For another instance, the segmentation algorithm may be a multi-task deep learning network model that uses synchronous detection and segmentation. Common deep learning networks such as Mask R-CNN, PolarMask, and SOLO may be adopted. These networks typically first localize the approximate position of the ROI and then perform fine segmentation on the ROI area.

For yet another instance, the segmentation algorithm may be a traditional image processing algorithm. Region-based segmentation algorithms may be used, including region growing, watershed, and Otsu's thresholding, among others. Gradient-based segmentation algorithms such as Sobel and Canny operators can also be employed.

Furthermore, the segmentation algorithm may be a machine learning segmentation model. This model is trained based on collected ultrasound images and their annotated results. Machine learning segmentation models such as SVM, Kmeans, and Cmeans may be utilized. The machine learning segmentation model performs binary classification on the grayscale or texture values of the pixels in the ultrasound image to determine whether each pixel or the texture feature vector representing the current pixel belongs to the left or right ventricular region, thereby achieving the extraction of the boundary contours of the left and right ventricles.

Third Approach: The processing unit 20 obtains the contours (boundaries) of the left and right ventricles based on the manual annotations made by the doctors on the ultrasound images.

After determining the contours of the left and right ventricles, the processing unit 20 may also display the ultrasound images marked with the contours of the left and right ventricles through the display unit 30. As shown in FIG. 6, multiple points are used to mark the left ventricular contour. This allows doctors to see if the identified contours are accurate and make manual adjustments if necessary.

The processing unit 20 may obtain the motion parameter of at least one myocardial segment of the left ventricle and the motion parameter of at least one myocardial segment of the right ventricle based on the contours of the left and right ventricles in the first ultrasound image data. For example, continuous tracking of the contours of the left and right ventricles may be performed to obtain the motion parameters of the myocardial segments; or, the motion parameters of the myocardial segments may be obtained based on the contours of the left and right ventricles corresponding to two or more frames of ultrasound image data in the first ultrasound image data. The following step 22 and step 23 are illustrated with continuous tracking as an example.

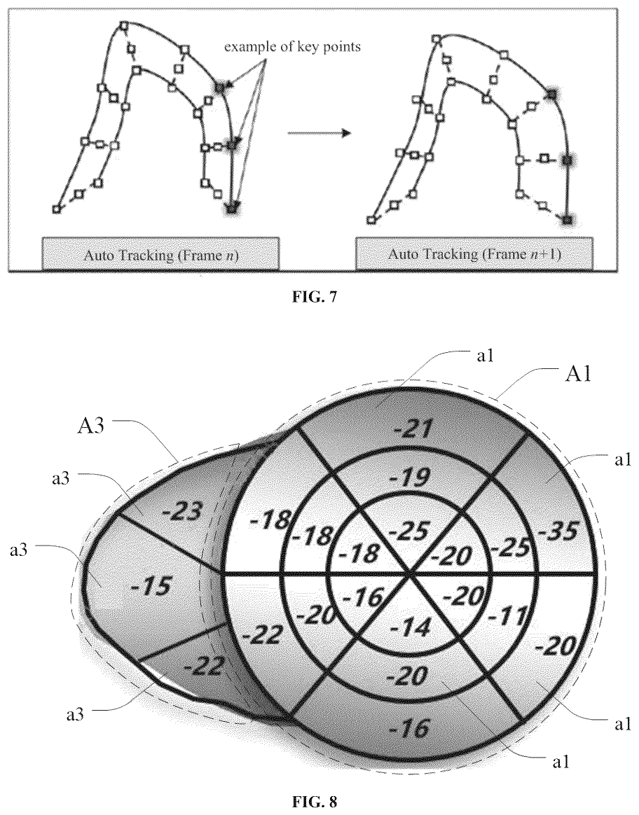

Step 22: the processing unit 20 tracks the left and right ventricular contours in the first ultrasound image data to obtain tracking results. Specifically, the tracking may involve monitoring multiple key points on the contours of the left and right ventricles. As shown in FIG. 7, the depicted points represent key points that collectively delineate the ventricular contours, meaning these key points are used to mark critical positional landmarks of the contours. The key points may be located on the endocardium, epicardium, or myocardium, as long as they can collectively identify the ventricular contour (the contours of the endocardium, epicardium, and myocardium may all be regarded as the ventricular contour).

Specifically, the processing unit 20 may adopt speckle tracking technology to track the position of the same key point (ultrasound scattering speckle) in each frame of the ultrasound image, thereby obtaining the positional changes of each key point. Since the cardiac imaging plane is known and the contours of the left and right ventricles have been identified in the ultrasound image, it is also known which myocardial segments the left and right ventricles contain in the ultrasound image. Based on the positions of the key points and myocardial segments, the myocardial segment where each key point is located may be determined. Thus, the processing unit 20 may determine the positional change relationship of the myocardial segments to which each key point belongs based on the positional changes of the key points.

When the motion displacement and deformation of myocardial segments are relatively small, it can be approximately assumed that the speckle patterns of the myocardial segments remain fixed. In ultrasound images, the motion of a specific myocardial segment may be tracked and quantitatively measured by tracking the movement of a specific speckle.

The processing unit 20 tracks the ultrasound speckles (key points) on the contours of the left and right ventricles (such as endocardium, epicardium or myocardial layer), obtaining the motion conditions (tracking results) of the myocardial segments (myocardial tissue structure) to which the ultrasound speckles belong, such as one or more of the velocity, displacement and deformation of the myocardial segments. Through these tracking results, the physiological characteristics of the cardiac tissue can be quantitatively analyzed.

Step 23: the processing unit 20 processes the tracking results to obtain the motion parameter of at least one myocardial segment of the left and right ventricles, typically obtaining the motion parameters of multiple myocardial segments of the left ventricle and the motion parameters of multiple myocardial segments of the right ventricle. The motion parameters may include strain, strain rate, velocity, displacement or contrast agent peak time. When the motion parameters include velocity, displacement, and contrast agent peak time, they are actually obtained in the tracking results of the previous step, so step 23 may not be necessary. This embodiment is illustrated with the motion parameters including strain or strain rate as an example.

The strain (Strain) may be calculated by the following formula:

Strain=(LES−LED)/LED; where LES is the length of a myocardial segment at the end of systole, and LED is the length of a myocardial segment at the end of diastole.

The strain rate (Strainrate) may be calculated by the following formula:

Strainrate=(Lt−Lt−1)/Lt*framerate; where Lt is the length of a myocardial segment in the t-th frame of ultrasound image from the ultrasound image data, Lt−1 is the length of a myocardial segment in the (t−1)-th frame of the ultrasound image from the ultrasound image data, and framerate is the frame rate of the ultrasound image data.

In patients with cardiovascular obstruction, regions supplied by the obstructed vessel may exhibit reduced motion amplitude compared to normally supplied areas. During cardiac motion, the affected area by the obstructed vessel demonstrates passive motion (movement induced by mechanical drag from adjacent tissues). Therefore, some of its motion parameters such as deformation, strain, and strain rate will be significantly abnormal. The speckle tracking technique can accurately measure the movement of the heart and calculate the motion parameters of different parts of the heart, enabling localization of abnormal motion patterns and providing clinically diagnostic significance for users. The quantitative analysis of cardiac motion using speckle tracking techniques can be used to assess global/regional ventricular/atrial function, diastolic/systolic performance, evaluate cardiac synchronization abnormalities, and subsequent follow-up treatment.

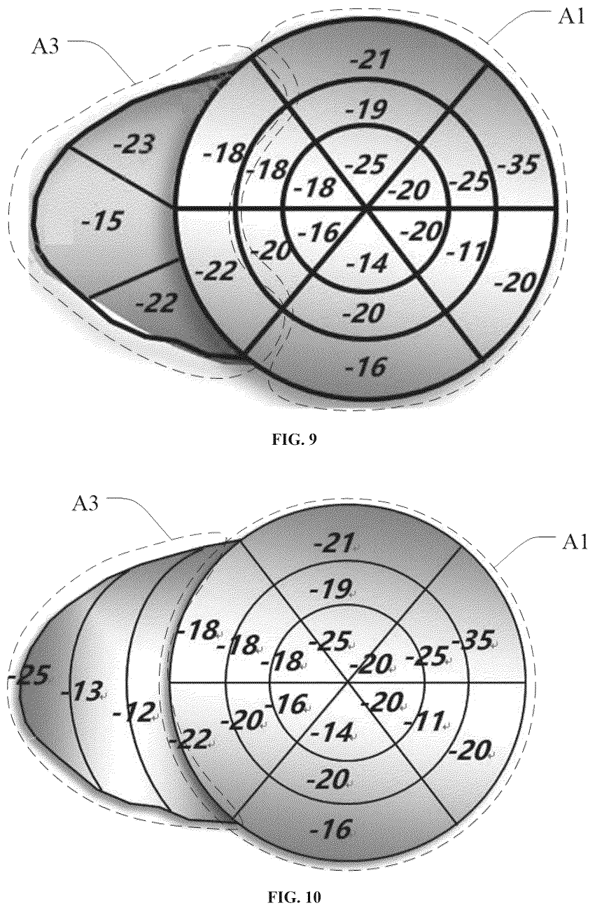

Step 3: the processing unit 20 displays the composite bull's-eye plot of the left and right ventricles through the display unit 30. For instance, upon receiving a display instruction, the processing unit 20 shows the composite bull's-eye plot of the left and right ventricles via the display unit 30. As illustrated in FIGS. 8-13, the composite bull's-eye plot comprises a left ventricular bull's-eye subplot A1 and the right ventricular bull's-eye subplot A3. The left ventricular bull's-eye subplot A1 includes a plurality of left ventricular myocardial regions a1 of the plurality of myocardial segments of the left ventricle. That is, one left ventricular myocardial region a1 represents one myocardial segment of the left ventricle. The right ventricular bull's-eye subplot A3 includes a plurality of right ventricular myocardial regions a3 corresponding to the plurality of myocardial segments of the right ventricle. That is, one right ventricular myocardial region a3 represents one myocardial segment of the right ventricle. In this embodiment, multiple means two or more.

Since the composite bull's-eye plot can present the myocardial strain of both the left and right ventricles, and there are shared myocardial segments between the left and right ventricles, the attribution of these shared myocardial segments may affect the specific composition of the left ventricular bull's-eye subplot A1 and the right ventricular bull's-eye subplot A3 (without affecting the composite bull's-eye plot). The following provides a detailed explanation of this.

In some embodiments, the right ventricular bull's-eye subplot A3 may include: the right ventricular myocardial region corresponding to myocardial segments of the right ventricle, excluding all the shared myocardial segments. That is, as shown in FIG. 8, the right ventricular bull's-eye subplot A3 does not include the myocardial regions corresponding to the shared myocardial segments. The two myocardial regions (with motion parameters of −18 and −22 respectively) adjacent to the right ventricular bull's-eye subplot A3 in FIG. 8 are the myocardial regions corresponding to the shared myocardial segments. All shared myocardial segments are allocated to the myocardial segments of the left ventricle. Since the bull's-eye plots in the existing technology include the myocardial segments of the left ventricle and the shared myocardial segments, this division method in this embodiment is more in line with the habits of doctors.

In some embodiments, the right ventricular bull's-eye subplot A3 may include: right ventricular myocardial region corresponding to myocardial segments of the right ventricle, excluding some of the shared myocardial segments. Correspondingly, the left ventricular bull's-eye subplot A1 includes: the left ventricular myocardial region corresponding to the myocardial segments of the left ventricle, excluding some of the shared myocardial segments. In other words, the right ventricular bull's-eye subplot A3 contains the myocardial regions corresponding to a part of the shared myocardial segments, and the left ventricular bull's-eye subplot A1 contains the myocardial regions corresponding to another part of the shared myocardial segments.

In some embodiments, the left ventricular bull's-eye subplot A1 may include: the left ventricular myocardial region corresponding to the myocardial segments of the left ventricle, excluding all the shared myocardial segments, that is, as shown in FIG. 9, the left ventricular bull's-eye subplot A1 does not include the myocardial regions corresponding to the shared myocardial segments, and all the shared myocardial segments are allocated to the myocardial segments of the right ventricle.

The motion parameters of at least one myocardial segment of the left ventricle are presented in the corresponding left ventricular myocardial region. Similarly, the motion parameters of at least one myocardial segment of the right ventricle are presented in the corresponding right ventricular myocardial region. That is, the motion parameters of the myocardial segments of the left and right ventricles obtained in step 2 may all be displayed in the myocardial region corresponding to the composite bull's-eye plot. In an ultrasound image of one cardiac imaging plane, the covered myocardial segments are limited, so the number of myocardial segments with motion parameters obtained in step 2 may be less than the myocardial regions (a1 and a3) in the composite bull's-eye plot, that is, some myocardial regions in the composite bull's-eye plot display motion parameters while others do not. Doctors can scan multiple cardiac imaging planes to obtain the corresponding first ultrasound image data, that is, steps 1 and 2 are repeated, so that motion parameters of more myocardial segments can be displayed in the composite bull's-eye plot in step 3. As shown in FIGS. 8-13, by repeatedly performing steps 1 and 2 multiple times, all myocardial regions in the composite bull's-eye plot in step 3 can display the motion parameters of the corresponding myocardial segments. Compared with the existing bull's-eye plot that only shows the information of the left ventricle, the composite bull's-eye plot in this embodiment expands more information, optimizes the operation and usage efficiency of doctors, and enables doctors to quickly understand the overall picture of a patient's cardiac contraction and relaxation functions.

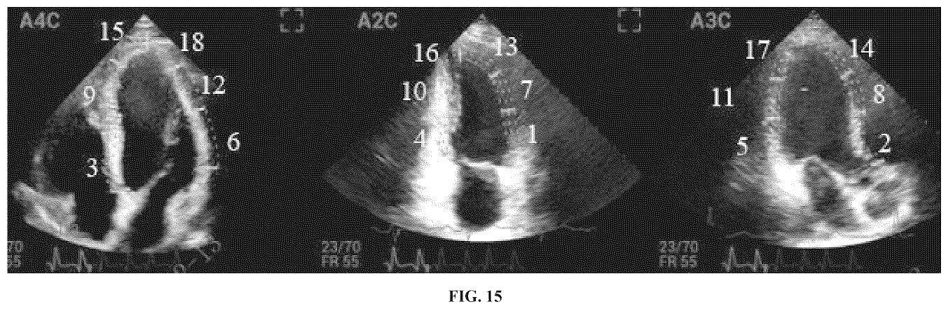

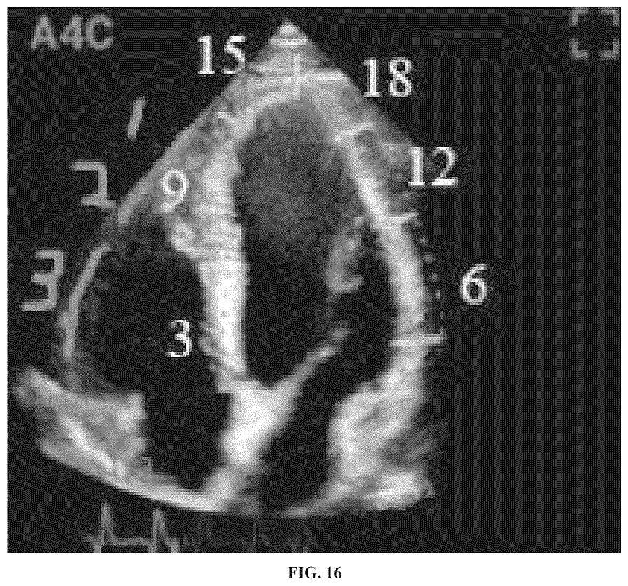

Considering the habits of doctors, the left ventricular myocardial regions in the left ventricular bull's-eye subplot A1 may include: 16 myocardial regions corresponding respectively to the 16 myocardial segments of the left ventricle, and the arrangement of these 16 myocardial regions may be as shown in the left figure of FIG. 14. The left ventricular myocardial regions in the left ventricular bull's-eye plot A1 may also include: 17 myocardial regions corresponding respectively to the 17 myocardial segments of the left ventricle, and the arrangement of these 17 myocardial regions may be as shown in the middle figure of FIG. 14. The left ventricular myocardial regions in the left ventricular bull's-eye plot A1 may also include: 18 myocardial regions corresponding respectively to the 18 myocardial segments of the left ventricle, and the arrangement of these 18 myocardial regions may be as shown in the right figure of FIG. 14. For the three cardiac imaging planes A4C, A2C and A3C, the myocardial segments from which motion parameters can be obtained based on the first ultrasound image data as shown in FIG. 15.

In some embodiments, the right ventricular bull's-eye subplot A3 may include one or more: a myocardial region corresponding to the apical segment of a right ventricular free wall, a myocardial region corresponding to the mid segment of a/the right ventricular free wall, and a myocardial region corresponding to the basal segment of the right ventricular free wall. This embodiment takes the case of including the myocardial regions corresponding to these three myocardial segments as an example for illustration. As shown in FIG. 16, when the cardiac imaging plane in step 1 is the A4C view, the motion parameters of these three myocardial segments of the right ventricle may be obtained in step 2 and all displayed in the right ventricular bull's-eye subplot A3.

In some embodiments, the positional relationship between the left ventricular bull's-eye subplot A1 and the right ventricular bull's-eye subplot A3 in the composite bull's-eye plot corresponds to the positional relationship between the left and right ventricles in a cardiac imaging plane (hereafter referred to as a “first imaging plane” for clarity). This cardiac imaging plane here may or may not be the same as the cardiac imaging plane referenced in step 1. The workflow illustrated in FIG. 3 may be executed multiple times, involving multiple cardiac imaging planes, and the cardiac imaging plane that embodies the positional relationship described above may be selected from among these multiple cardiac imaging planes. This configuration enables doctors to correlate motion parameters displayed in the left and right ventricular bull's-eye subplots with the myocardial segments of the left and right ventricles.

The specific structure of the composite bull's-eye plot may vary across different embodiments. Some configurations may incorporate the interventricular septum region A2, while others may exclude the interventricular septum region A2. Exemplary implementations are described as follows.

In FIGS. 8-10, the composite bull's-eye plot exclusively comprises the left ventricular bull's-eye subplot A1 and the right ventricular bull's-eye subplot A3, while excluding the interventricular septum region A2. That is, in the cardiac imaging plane (the first imaging plane) that shows the positional relationship between the left ventricular bull's-eye subplot A1 and the right ventricular bull's-eye subplot A3, the interventricular septum is not visible. The left ventricular bull's-eye subplot A1 and the right ventricular bull's-eye subplot A3 are adjacent and may be arranged left and right or right and left. As shown in FIGS. 8 and 9, the various right ventricular myocardial regions a3 of the right ventricular bull's-eye subplot A3 may be arranged in sequence from top to bottom along a curve (such as an arc). If in a cardiac imaging plane, the apical segment of the right ventricular free wall, the mid segment of the right ventricular free wall, and the basal segment of the right ventricular free wall are arranged in sequence from top to bottom along a curve, then the motion parameters of the upper, middle and lower myocardial regions a3 in A3 are the motion parameters of the apical segment, mid segment and basal segments of the right ventricle free wall. As shown in FIG. 10, the various right ventricular myocardial regions a3 of the right ventricle bull's-eye subplot A3 may also be arranged in sequence from left to right.

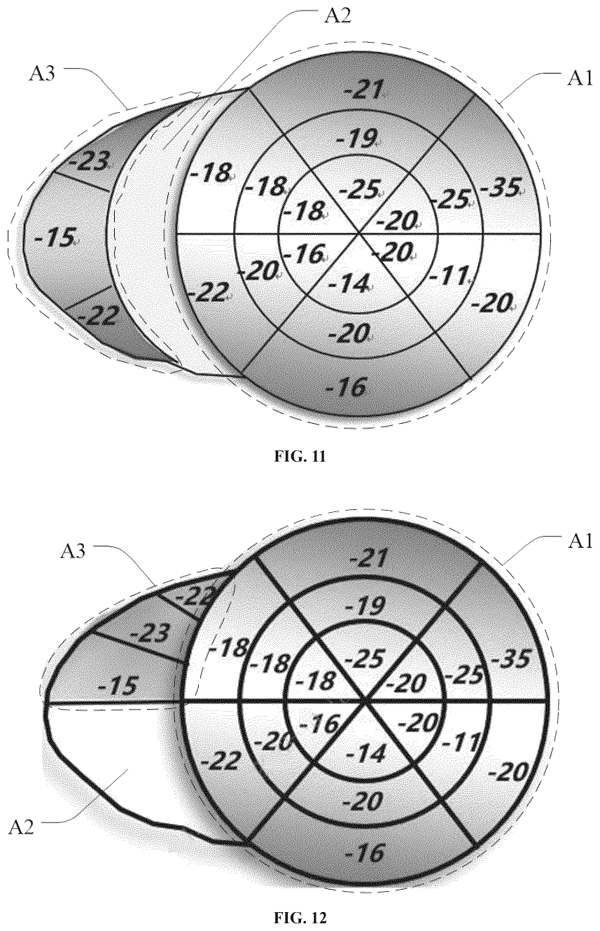

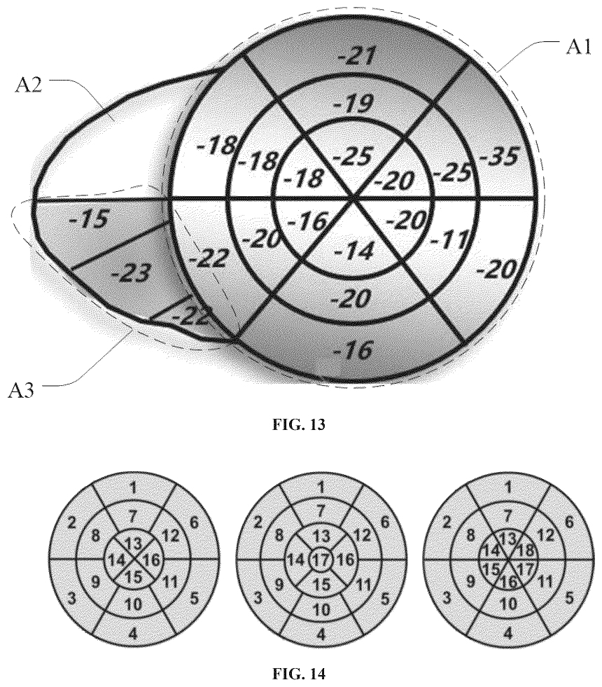

In FIGS. 11-13, the composite bull's-eye plot also includes the interventricular septum region A2 for presenting the interventricular septum, the interventricular septum region A2 is adjacent to the left ventricular bull's-eye subplot A1 and/or the right ventricular bull's-eye subplot A3. As shown in FIG. 11, the right ventricular bull's-eye subplot A3, the interventricular septum region A2, and the left ventricular bull's-eye subplot A1 may be arranged from left to right or from right to left in sequence. As shown in FIGS. 12 and 13, the right ventricular bull's-eye subplot A3 and the interventricular septum region A2 are arranged vertically and both are adjacent to the left ventricular bull's-eye subplot A1. In FIGS. 11-13, the respective right ventricular myocardial regions of the right ventricular bull's-eye subplot A3 are arranged vertically in sequence along a curve (such as an arc).

In some embodiments, the left ventricular bull's-eye subplot A1 may be a traditional left ventricular bull's-eye plot, which will not be elaborated here.

It can be seen that the composite bull's-eye plot provided by the present disclosure can present the strain of multiple myocardial segments of the left and right ventricles, facilitating doctors to comprehensively understand the overall condition of the patient's ventricles.

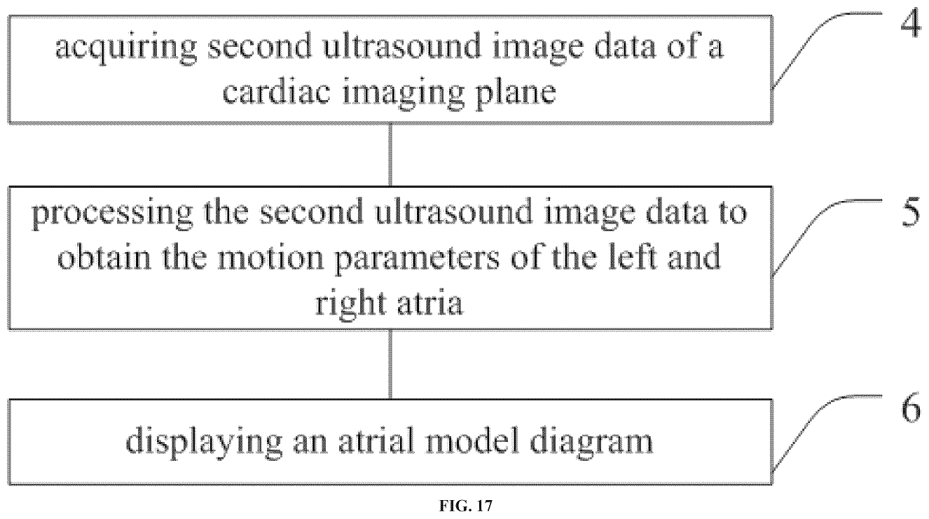

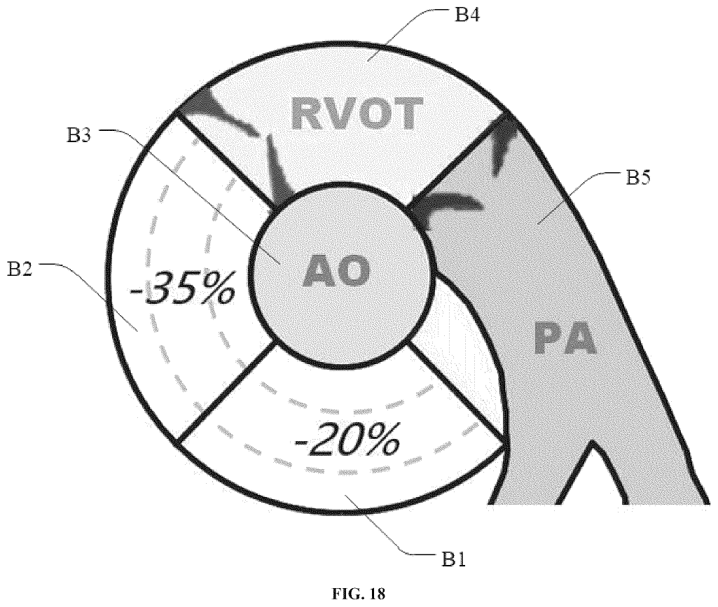

The present disclosure can also present the motion parameters of the atrium, and the specific process thereof is shown in FIG. 17 and includes the following steps:

Step 4: the processing unit 20 acquires second ultrasound image data of a cardiac imaging plane through the data acquisition unit 10. The second ultrasound image data contains image data of the left atrium of a heart and image data of the right atrium of the heart. Similar to the first ultrasound image data, the second ultrasound image data is mainly ultrasound video data, and the source is the same as that of the first ultrasound image data. For details, please refer to the previous embodiment, and no further elaboration is provided here. If a cardiac imaging plane contains both the left and right atria and the left and right ventricles, then the first and second ultrasound image data can be the same ultrasound image data, that is, steps 1 and 4 can be the same step.

Step 5: the processing unit 20 processes the second ultrasound image data to obtain the motion parameters of the left atrium and the motion parameters of the right atrium. The motion parameters of the left and right atria may be the overall motion parameters of the left and right atria; or, as shown in the example of FIG. 3, the motion parameters of multiple myocardial segments. This embodiment takes the former as an example for explanation. Specifically, the processing unit 20 can determine the contours of the left and right atria in the second ultrasound image data, and then track the contours of the left and right atria in the second ultrasound image data to obtain tracking results. The tracking results may be processed to obtain the motion parameters of the left and right atria. The specific process is the same as that of step 2 in the aforementioned embodiments, except that the left and right ventricles are replaced with the left and right atria. Accordingly, it will not be elaborated here. Similarly, the motion parameters may include strain, strain rate, velocity, displacement, or contrast agent peak time, etc. This embodiment takes strain as an example for explanation.

Step 6: the processing unit 20 displays an atrial model diagram via the display unit 30. For instance, after receiving a display instruction, the processing unit 20 displays the atrial model diagram through the display unit 30. As shown in FIG. 18, the atrial model diagram is used to display the motion parameters of the left atrium and the motion parameters of the right atrium. In this way, doctors can also observe the strain conditions of the left and right atria, enabling them to better analyze a patient.

The atrial model diagram may include: a left atrial region B1 corresponding to the left atrium, a right atrial region B2 corresponding to the right atrium, and an aortic region B3 corresponding to an aorta (AO). That is, the left atrial region B1 represents the left atrium, the right atrial region B2 represents the right atrium, and the aortic region B3 represents the aorta. The motion parameters of the left atrium are displayed in the left atrial region B1, and the motion parameters of the right atrium are displayed in the right atrial region B2.

In some embodiments, the atrial model diagram may further include: a right ventricular outflow tract (RVOT) region B4 corresponding to the right ventricular outflow tract and a pulmonary artery (PA) region B5 corresponding to the pulmonary artery. That is, the right ventricular outflow tract region B4 represents the right ventricular outflow tract, and the pulmonary artery region B5 represents the pulmonary artery. In the atrial model diagram, the positional relationship among the left atrial region B1, the right atrial region B2, the aortic region B3, the right ventricular outflow tract region B4, and the pulmonary artery region B5 is consistent with the positional relationship of the corresponding left atrium, right atrium, aorta, right ventricular outflow tract, and pulmonary artery in a cardiac imaging plane (which may be referred to as a second imaging plane for ease of distinction). This cardiac imaging plane can be the cardiac imaging plane in step 4 or not. That is, the arrangement of the various regions in the atrial model diagram reflects the positions of the corresponding anatomical structures in the cardiac imaging plane, making it very intuitive for doctors to quickly match the strain values in the diagram with their respective atria.

In some short-axis sections of the heart, the arrangement of the left atrium, right atrium, aorta, right ventricular outflow tract and pulmonary artery is similar to that shown in FIG. 18. Therefore, in the corresponding atrial model diagram, the aortic region B3 is located in the center, and the left atrial region B1, right atrial region B2, right ventricular outflow tract region B4 and pulmonary artery region B5 are arranged in sequence around the aortic region B3. This not only shows the strain of the left and right atria, but also vividly presents the positional relationship of the main anatomical structures in the cardiac imaging plane.

The composite bull's-eye plot and the atrial model diagram may be displayed separately or simultaneously on the same display interface. In this way, doctors can view the strain conditions of all four chambers of the heart, which is very convenient. Whether to display one or both of the composite bull's-eye plot and the atrial model diagram may be determined by a doctor's operation. For example, the processing unit 20 receives a selection instruction issued by a user and displays the corresponding composite bull's-eye plot and/or atrial model diagram based on the selection instruction. In this embodiment, after receiving the display instruction, the composite bull's-eye plot is displayed by default. After the processing unit 20 receives a first switching instruction issued by the user, it switches the displayed composite bull's-eye plot on the display interface to the atrial model diagram in response to the first switching instruction. That is, the composite bull's-eye plot may be displayed to meet the general needs of doctors, and when doctors need more strain information, they can issue the first switching instruction.

As can be seen, the method for presenting cardiac myocardial strain and the corresponding system provided by the present disclosure operate as follows: by acquiring ultrasound image data from multiple cardiac chambers, the system processes data of the same case across multiple chambers and/or imaging planes to obtain myocardial speckle tracking traces in the ultrasound images and performs tissue motion tracking, thereby calculating strain values; constructs one or more correlation charts reflecting multi-chamber strain patterns; and dynamically combines or independently displays these charts based on user selections. This intelligent and efficient clinical auxiliary analysis tool enhances both diagnostic efficiency and accuracy for doctors.

Through the above-mentioned composite bull's-eye plot and/or atrial model diagram, more information has been added on the basis of the existing single left ventricular strain bull's-eye plot, optimizing the operation and usage efficiency for doctors and enabling them to quickly understand the overall picture of a patient's cardiac contraction and relaxation functions.

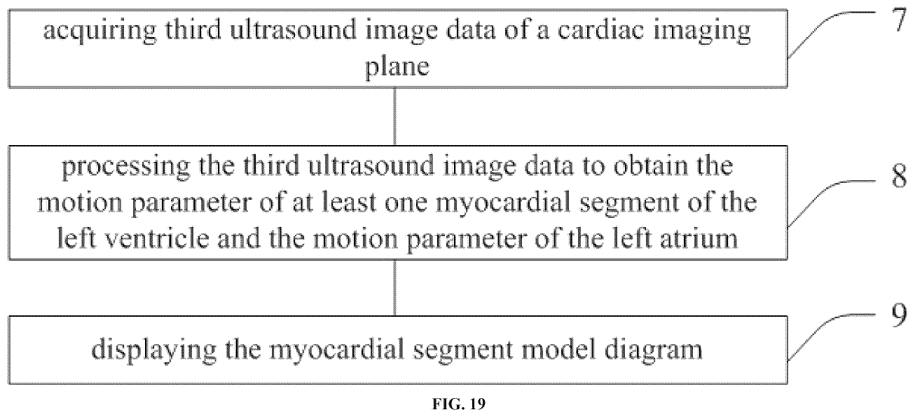

In addition to the aforementioned composite bull's-eye plot and atrial model diagram, the present disclosure also provides another type of diagram that can present the motion parameters of the myocardium of multiple chambers of a heart. See FIG. 19 for details. The process may include the following steps:

Step 7: the processing unit 20 acquires third ultrasound image data of a cardiac imaging plane by the data acquisition unit 10. The third ultrasound image data contains image data of at least one myocardial segment of the left ventricle and image data of the left atrium. Similar to the first ultrasound image data, the third ultrasound image data is mainly ultrasound video data, and the its acquisition is the same as that of the first ultrasound image data. For details, see the previous embodiments, which will not be repeated here. If a cardiac imaging plane contains both the left and right ventricles and the left atrium, the first and third ultrasound image data may be the same ultrasound image data, and steps 1 and 7 may be the same step.

Step 8: the processing unit 20 processes the third ultrasound image data to obtain the motion parameters of at least one myocardial segment of the left ventricle and the motion parameters of the left atrium. The motion parameters of the left atrium may be the overall motion parameters of the left atrium or the motion parameters of multiple myocardial segments of the left atrium. This embodiment takes the former as an example for illustration. Specifically, the processing unit 20 can determine the contour of the left ventricle and the contour of the left atrium in the third ultrasound image data, and then obtain the motion parameters of at least one myocardial segment of the left ventricle and the motion parameters of the left atrium based on the contour of the left ventricle and the contour of the left atrium in the third ultrasound image data. The specific process is the same as that of step 2 in the previous embodiments, except that the right ventricle is changed to the left atrium. If the overall motion parameters of the left atrium are obtained, it can be considered that the left atrium has only one myocardial segment. Therefore, the specific method for obtaining the motion parameters in step 2 of the previous embodiments is also applicable to other embodiments and will not be repeated here. Similarly, the motion parameters can include strain, strain rate, velocity, displacement, or contrast agent peak time, etc. This embodiment takes strain as an example for illustration.

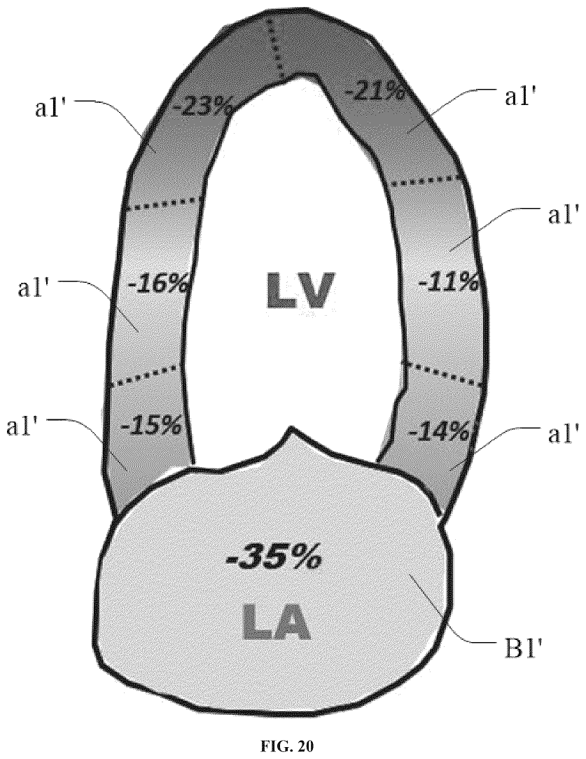

Step 9: the processing unit 20 displays the myocardial segment model diagram by the display unit 30. For example, after receiving a display instruction, the processing unit 20 displays the myocardial segment model diagram through the display unit 30. As shown in FIGS. 20-22, the myocardial segment model diagram can display at least the motion parameters of multiple myocardial segments of the left ventricle (LV) and the motion parameters of the left atrium (LA), so that doctors can observe the strain conditions of the left ventricle and left atrium and better analyze the patient. The myocardial segment model diagram includes: a plurality of left ventricular myocardial regions a1′ corresponding to multiple myocardial segments of the left ventricle (LV) and a left atrial region B1′ corresponding to the left atrium (LA). Similarly, one left ventricular myocardial region a1′ represents one myocardial segment of the left ventricle. The motion parameters of the myocardial segments of the left ventricle obtained in step 8 are presented in the corresponding left ventricular myocardial regions a1′, and the left atrial region B1′ displays the motion parameters of the left atrium.

The myocardial segment model diagram may take various forms. FIGS. 20-22 show three types. The following provides explanations for each of these three myocardial segment model diagrams.

The myocardial segment model diagram shown in FIG. 20 is referred to as the first myocardial segment model diagram for ease of distinction. It includes multiple left ventricular myocardial regions a1′ corresponding to multiple myocardial segments of the left ventricle (LV) and a left atrial region B1′ corresponding to the left atrium (LA), but may not include the right ventricular region, right ventricular myocardial region, right atrial region, and aortic region, etc. In this first myocardial segment model diagram, the multiple left ventricular myocardial regions a1′ are arranged to form the contour of the left ventricle in a cardiac imaging plane; the positional relationship between each left ventricular myocardial region a1′ and the left atrial region B1′ corresponds to (e.g. being in consistent with) the positional relationship between the corresponding left ventricular myocardial segments and the left atrium in a cardiac imaging plane (referred to as the third imaging plane for case of distinction), that is, the positional distribution (arrangement) of each left ventricular myocardial region a1′ and the left atrial region B1′ in the first myocardial segment model diagram reflects the anatomical structure of each left ventricular myocardial segment and the left atrium in the third imaging plane. The third imaging plane may be the cardiac imaging plane in step 7 or not. The multiple left ventricular myocardial regions a1′ form the contour of the left ventricle and may be located above or below the left atrial region B1′ (above in FIG. 20).

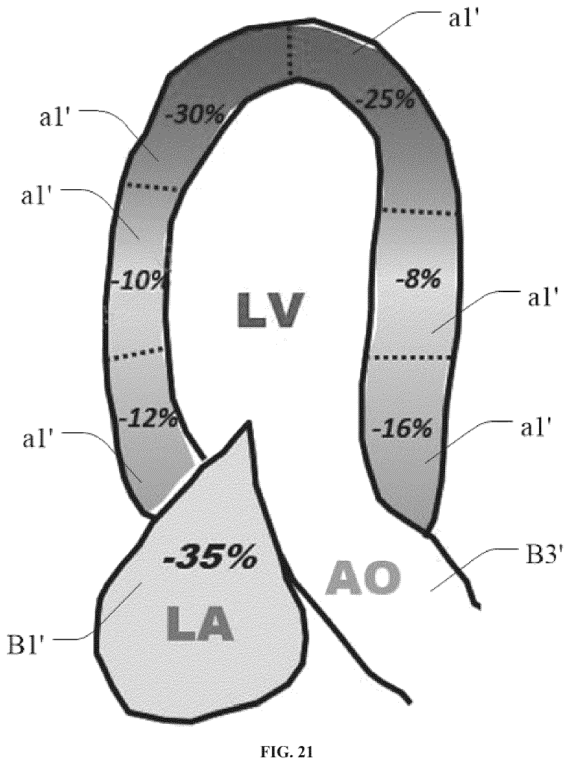

The myocardial segment model diagram shown in FIG. 21 is referred to as a second myocardial segment model diagram for ease of distinction. It includes multiple left ventricular myocardial regions a1′ corresponding to multiple myocardial segments of the left ventricle (LV), a left atrial region B1′ corresponding to the left atrium (LA), and an aortic region B3′ corresponding to the aorta (AO), but may not include the right ventricular region, right ventricular myocardial region, and right atrial region, etc. The third ultrasound image data may or may not contain image data of the aorta of the heart. In this second myocardial segment model diagram, the multiple left ventricular myocardial regions a1′ are arranged to form the contour of the left ventricle in a cardiac imaging plane. The positional relationship between each of the multiple left ventricular myocardial regions a1′, the left atrial region B1′, and the aortic region B3′ in the second myocardial segment model diagram corresponds to the positional relationship between the corresponding left ventricle, left atrium, and aorta in a cardiac imaging plane (referred to as a fourth imaging plane for ease of distinction). The left atrial region B1′ and the aortic region B3′ may be located above or below the contour of the left ventricle formed by the multiple left ventricular myocardial regions a1′ (below in FIG. 21).

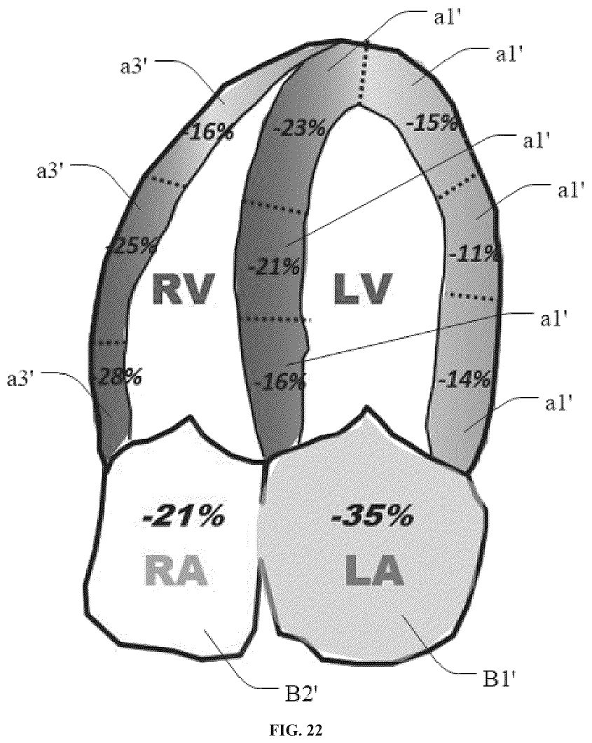

The myocardial segment model diagram shown in FIG. 22 is referred to as a third myocardial segment model diagram for case of distinction. The third ultrasound image data in step 7 may also include image data of at least one myocardial segment of the right ventricle and image data of the right atrium of a heart. Correspondingly, in step 8, the processing unit 20 can process the third ultrasound image data to obtain the motion parameters of at least one myocardial segment of the right ventricle and the motion parameters of the right atrium. The motion parameters of the right atrium can be the motion parameters of the entire right atrium or the motion parameters of multiple myocardial segments of the right atrium. This embodiment takes the former as an example for explanation. Specifically, the processing unit 20 can determine the contour of the right ventricle and the contour of the right atrium in the third ultrasound image data, and then obtain the motion parameters of at least one myocardial segment of the right ventricle and the motion parameters of the right atrium based on the contours of the right ventricle and the right atrium in the third ultrasound image data. The specific process is the same as that in step 2 of the aforementioned embodiment, except that the left ventricle is replaced with the right atrium. If the motion parameters of the entire right atrium are obtained, it can be considered that the right atrium has only one myocardial segment. Therefore, the specific method for obtaining the motion parameters in step 2 of the aforementioned embodiment is also applicable to other embodiments and will not be elaborated here. Similarly, the motion parameters can include strain, strain rate, velocity, displacement or contrast agent peak time, etc. This embodiment takes strain as an example for illustration.

Correspondingly, the third myocardial segment model diagram also includes: multiple right ventricular myocardial regions a3′ corresponding to multiple myocardial segments of the right ventricle, and a right atrial region B2′ corresponding to the right atrium. Similarly, a right ventricular myocardial region a3′ represents a myocardial segment of the right ventricle, and the right atrial region B2′ represents the right atrium. The motion parameters of at least one myocardial segment of the right ventricle are presented in the corresponding right ventricular myocardial region a3′; the right atrial region B2′ shows the motion parameters of the right atrium. In the third myocardial segment model diagram, the various left ventricular myocardial regions a1′ are arranged to form the contour of the left ventricle in a cardiac imaging plane, and the various right ventricular myocardial regions a3′ are arranged to form the contour of the right ventricle in a cardiac imaging plane. The positional relationship among the various left ventricular myocardial regions a1′, the left atrial region B1′, the various right ventricular myocardial regions a3′, and the right atrial region B2′ in the third myocardial segment model diagram is consistent with the positional relationship among the corresponding left ventricle, left atrium, right ventricle, and right atrium on a cardiac imaging plane (for ease of distinction, referred to as a fifth imaging plane), that is, the positional distribution (arrangement) of the various left ventricular myocardial regions a1′, the left atrial region B1′, the various right ventricular myocardial regions a3′, and the right atrial region B2′ in the third myocardial segment model diagram reflects the anatomical structure of the left ventricle, left atrium, right ventricle, and right atrium in the imaging plane. The imaging plane can be the cardiac imaging plane in step 7 or not. The various left ventricular myocardial regions a1′ form the contour of the left ventricle and can be located above or below the left atrial region B1′ (above in FIG. 22), and similarly, the various right ventricular myocardial regions a3′ form the contour of the right ventricle and can be located above or below the right atrial region B2′ (above in FIG. 22).

For the division of the shared myocardial segments between the left and right ventricles, it can be carried out as shown in the example of FIG. 3, where all the shared myocardial segments are assigned to the left ventricle, some are assigned to the left ventricle and the rest to the right ventricle, or all are assigned to the right ventricle.

The right ventricular myocardial region A3′ in the third myocardial segment model diagram may include one or more of a myocardial region corresponding to the apical segment of the right ventricular free wall, a myocardial region corresponding to the mid segment of the right ventricular free wall, and a myocardial region corresponding to the basal segment of the right ventricular free wall. Each right ventricular myocardial region a3′ may be arranged in sequence along a curve (such as an arc) from top to bottom, thereby forming the contour of the right ventricle together with the myocardial regions corresponding to the shared myocardial segments. In the first, second and third myocardial segment model diagrams, the left ventricular myocardial region a1′ may include the myocardial regions corresponding to a part of the 16, 17 or 18 myocardial segments of the left ventricle. Each left ventricular myocardial region a1′, together with the myocardial regions corresponding to the shared myocardial segments, forms the contour of the left ventricle.

In some embodiments of the composite bull's-eye plot, the first imaging plane that shows the positional relationship between the left and right ventricles can be a short-axis section. In some embodiments of the atrial model diagram, the second imaging plane that shows the positional relationship among the left and right atria, the aorta, the right ventricular outflow tract, and the pulmonary artery can be a short-axis section. In some embodiments of the myocardial segment model diagram, the third, fourth, and fifth imaging planes that show the positional relationship among the left ventricle, the left atrium, the right ventricle, the right atrium, and the aorta, etc., can be a long-axis section.

The myocardial segment model diagram displayed by the display unit 30 may be one or more of the first, second and third myocardial segment model diagrams. Considering any one of the first, second and third myocardial segment model diagrams, the myocardial segments represented by the myocardial regions included in it may only be a part of the myocardial segments divided in the ventricle. For instance, the left ventricle is divided into 18 myocardial segments, and each of the first, second and third myocardial segment model diagrams only shows the myocardial regions of 6 myocardial segments; accordingly, three myocardial segment model diagrams are needed to present the motion parameters of these 18 myocardial segments. Thus, in some embodiments, the myocardial segment model diagram displayed by the display unit 30 includes the first, second and third myocardial segment model diagrams, and the myocardial segments represented by the myocardial regions a1′ among these three myocardial segment model diagrams are all different. In this way, doctors can comprehensively grasp the motion parameters of each myocardial segment by integrating the motion parameters on these three myocardial segment model diagrams, just like a composite bull's-eye plot. Similarly, steps 7 and 8 can be repeated to obtain the third ultrasonic image data of different cardiac imaging planes each time and process it to obtain the corresponding motion parameters, so that the myocardial segment model diagram displayed in step 9 can show the corresponding motion parameters for each myocardial region, left and right atrial regions, etc.

The composite bull's-eye plot and the myocardial segment model diagram can be displayed separately or simultaneously on the same display interface. Whether to display one or both of the composite bull's-eye plot and the myocardial segment model diagram can be determined by a doctor's operation. For example, the processing unit 20 receives a selection instruction issued by a user and displays the composite bull's-eye plot and/or the myocardial segment model diagram corresponding to the selection instruction. In this embodiment, after receiving the display instruction, the composite bull's-eye plot is displayed by default. After the processing unit 20 receives a second switching instruction issued by the user, it responds to the second switching instruction and switches the displayed composite bull's-eye plot on the display interface to the myocardial segment model diagram (displaying one or more of the first, second and third myocardial segment model diagrams).

The atrial model diagram and the myocardial segment model diagram can be displayed separately or on the same display interface. Whether to display one or both of the atrial model diagram and the myocardial segment model diagram can be determined by the doctor's operation. For example, the processing unit 20 receives a selection instruction issued by the user and displays the atrial model diagram and/or the myocardial segment model diagram corresponding to the selection instruction. When one of the diagrams is displayed, it can be switched through operation. For example, after the processing unit 20 receives a third switching instruction issued by the user, it responds to the third switching instruction and switches the displayed atrial model diagram on the display interface to the myocardial segment model diagram.

The composite bull's-eye plot, the atrial model diagram and the myocardial segment model diagram can be displayed separately or on the same display interface. Whether to display one or more of the composite bull's-eye plot, the atrial model diagram and the myocardial segment model diagram can be determined by the doctor's operation. For example, the processing unit 20 receives a selection instruction issued by the user and displays one or more of the composite bull's-eye plot, the atrial model diagram and the myocardial segment model diagram corresponding to the selection instruction. In this embodiment, after receiving the display instruction, the composite bull's-eye plot is displayed by default. After the processing unit 20 receives the selection instruction issued by the user, it switches the displayed composite bull's-eye plot on the display interface to the atrial model diagram or the myocardial segment model diagram corresponding to the selection instruction in response to the selection instruction.

The motion parameters in the composite bull's-eye plot, the atrial model diagram and/or the myocardial segment model diagram can be modified by doctors. After the processing unit 20 receives the instruction of selecting a region and the motion parameters input by the user, it replaces the original motion parameters of the selected region with the input motion parameters. The selected region is an area selected by the user from the above-mentioned left and right ventricular myocardial regions and left and right atrial regions.

Doctors can even input the motion parameters of various regions and present them in the composite bull's-eye plot, the atrial model diagram, and/or the myocardial segment model diagram. For instance, the processing unit 20 receives multiple motion parameters of myocardial segments input by the user. After receiving a display instruction, it displays the composite bull's-eye plot, the atrial model diagram, and/or the myocardial segment model diagram, with the motion parameters input by the user presented in the corresponding regions of these diagrams. The processing unit 10 can also receive multiple motion parameters of myocardial segments, left atrium, and right atrium input by the user. After receiving a display instruction, it displays the composite bull's-eye plot, the atrial model diagram, and/or the myocardial segment model diagram, with the motion parameters input by the user presented in the corresponding regions of these diagrams.

The above-mentioned cardiac ultrasound auxiliary analysis system can be applied in one or more devices, such as mobile phones, various types of computers (such as tablets, laptops, PCs, etc.), servers, and ultrasound imaging equipment. In other words, mobile phones, various types of computers, servers, and ultrasound imaging equipment can all realize the above-mentioned functions of the cardiac ultrasound auxiliary analysis system.

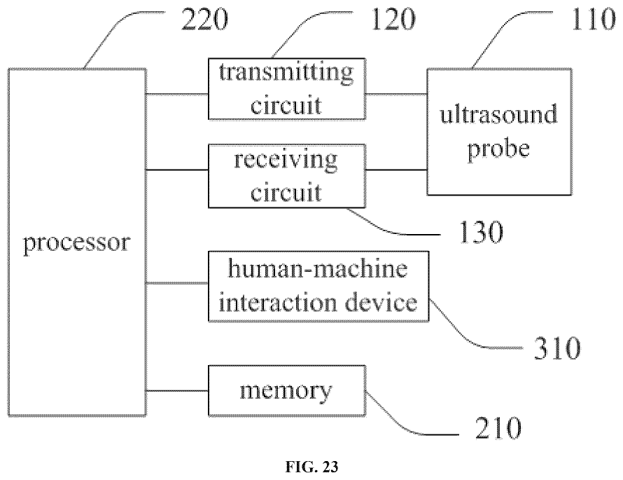

This embodiment is illustrated by taking an ultrasound imaging apparatus as an example. As shown in FIG. 23, the ultrasound imaging apparatus includes: an ultrasound probe 110, a transmitting circuit 120, a receiving circuit 130, a human-machine interaction device 310, a memory 210 and a processor 220.

The ultrasound probe 110 includes a transducer composed of multiple array-arranged elements (not shown in the figure). The elements are used to emit ultrasound waves according to the excitation electrical signal or convert the received ultrasound waves into electrical signals. Therefore, each element can be used to achieve the mutual conversion between electrical pulse signals and ultrasound waves, thereby enabling the emission of ultrasound waves to the biological tissue of a target object and also being capable of receiving the ultrasound echoes reflected back by the tissue.

The transmitting circuit 120 is used to excite the ultrasound probe 110 to emit ultrasound waves, for example, according to the control of the processor 220, to excite the ultrasound probe 110 to emit ultrasound waves to the target object (such as the heart).

The receiving circuit 130 is used to control the ultrasound probe 110 to receive the ultrasound echoes, for example, to receive the ultrasound echoes returned from the target object through the ultrasound probe 110 to obtain the ultrasound echo signals, and can also process the ultrasound echo signals. The receiving circuit 130 may include one or more amplifiers, analog-to-digital converters (ADC), etc.

The human-machine interaction device 310 is used for human-machine interaction, for example, to output visual information and receive user input. The human-machine interaction device 310 includes an input device and at least one display. The input device is used to receive user input and can adopt a keyboard, operation buttons, mouse, trackball, touchpad, etc., or can adopt a touch screen integrated into the display.

The memory 210 is used to store various types of data, such as ultrasound image data, programs for the processor 220 to execute, etc.

The processor 220 is used to execute the programs in the memory 210 to implement the functions of the above-mentioned processing unit 20, that is, under the control of the processor 220, the ultrasound imaging apparatus can execute the above method for presenting cardiac myocardial strain.

The ultrasound probe 110, the transmitting circuit 120 and the receiving circuit 130 can perform the function of the aforementioned data acquisition unit 10, for example, in steps 1, 4 and 7, the processor 220 controls the ultrasound probe 110 to emit ultrasound waves to the patient's heart through the transmitting circuit 120, and controls the ultrasound probe 110 to receive the echo of the ultrasound waves through the receiving circuit 130, obtaining the ultrasound echo signal, and processes the ultrasound echo signal to obtain the first, second and/or third ultrasound image data.

The human-machine interaction device 310 can perform the function of the aforementioned display unit 30, for example, in steps 3, 6 and 9, the processing unit 20 receives various instructions (such as display instructions, switching instructions, input operations, etc.) through the human-machine interaction device 310, and displays the composite bull's-eye plot, the atrial model diagram and the myocardial segment model diagram, etc. through the display of the human-machine interaction device 310.

The cardiac imaging planes of the ultrasound image data obtained in steps 1, 4 and 7 can be the sections of an echocardiogram examination. Thus, during the echocardiogram examination or while the examination is being conducted, a display instruction can be issued at any time to show one or more of the composite bull's-eye plot, the atrial model diagram and the myocardial segment model diagram. That is, the motion parameters of each region are collected during the echocardiogram examination, and subsequently, only the composite bull's-eye plot, the atrial model diagram and the myocardial segment model diagram need to be displayed directly. Specifically, after the processor 220 receives an echocardiogram examination instruction from the user through the human-machine interaction device, it receives the cardiac imaging plane selected by the user from the sections of the echocardiogram examination through the human-machine interaction device, and then controls the ultrasonic probe 110 to emit ultrasound waves to the selected cardiac imaging plane through the transmission circuit 120, and controls the ultrasonic probe 110 to receive the echo of the ultrasound waves through the receiving circuit 130 to obtain the ultrasound echo signal, processes the ultrasound echo signal to obtain the ultrasound image data and display it. The ultrasound image data is saved based on the image saving instruction issued by the user. Then, the user can select other cardiac imaging planes for the echocardiogram examination and repeat the process to conduct the echocardiogram examination. In fact, this is also repeating one or more of the aforementioned steps 1, 4 and 7. After the echocardiogram examination is completed or during the examination, one or more of the aforementioned steps 2, 5 and 8 can be executed. Before or after executing the aforementioned steps 2, 5 and 8, a display instruction issued by the user is received to execute one or more of the aforementioned steps 3, 6 and 9. In this way, obtaining the composite bull's-eye plot, the atrial model diagram and the myocardial segment model diagram requires almost no additional operation from the doctor, which is very convenient.

It can be seen that the solution provided by the above-mentioned embodiments of the present disclosure enables doctors to observe the myocardial strain conditions of multiple chambers, allowing them to simultaneously evaluate the strain analysis results of multiple chambers of the same patient. This enables doctors to make comprehensive judgments based on a single analysis, thereby improving their work efficiency.

The present disclosure refers to various exemplary embodiments for illustrative purposes. However, those skilled in the art will recognize that modifications and alterations may be made to these embodiments without departing from the scope of the disclosure. For instance, individual operational steps and components for performing such steps may be implemented in diverse manners depending on specific applications or considerations of cost functions associated with system operations (e.g., one or more steps may be deleted, modified, or consolidated with other steps).

Moreover, as understood by those skilled in the art, the principles disclosed may be embodied in a computer program product stored on a non-transitory computer-readable storage medium preloaded with computer-readable program code. Any tangible, non-transitory computer-readable storage medium may be utilized, including but not limited to: magnetic storage devices (e.g., hard disks, floppy disks); optical storage devices (e.g., CD-ROMs, DVDs, Blu-ray discs); and flash memory devices. The computer program instructions may be loaded onto a general-purpose computer, special-purpose computer, or other programmable data processing apparatus to create a machine, such that the instructions executed on the computer or programmable apparatus produce means for implementing specified functions. These instructions may also reside in a computer-readable memory, directing the computer or programmable apparatus to operate in a defined manner, thereby forming an article of manufacture comprising functional implementation means. Furthermore, the computer program instructions may be executed on a computer or programmable data processing apparatus to generate a computer-implemented process, wherein the executed instructions provide steps for realizing the specified functionality, including but not limited to: technical improvements in data processing efficiency (e.g., optimized memory allocation); and enhanced accuracy in algorithmic execution (e.g., reduced error margins in machine learning models).

While the principles disclosed herein have been illustrated through various embodiments, it should be understood that structural configurations, material selections, and component proportions particularly suited to specific operational environments may be modified without departing from the scope and spirit of the disclosure. Such modifications, along with other adaptations or adjustments, shall be encompassed within the scope of the present disclosure.

The foregoing detailed description has been described with reference to various embodiments. However, those skilled in the art will recognize that modifications and variations may be made without departing from the scope of the disclosure. Accordingly, the description of the disclosure shall be interpreted in an illustrative rather than restrictive sense, and all such modifications are intended to be included within its scope. Similarly, discussions of advantages, alternative solutions to problems, and operational benefits associated with the embodiments are provided above. Nevertheless, benefits, advantages, solutions to problems, and any elements that may produce such effects or render them more explicit shall not be construed as critical, required, or essential. Furthermore, the term ‘coupled’ and its derivatives encompass physical connections (e.g., mechanical joints), electrical connections (e.g., circuit interconnects), magnetic linkages (e.g., inductive coupling), optical interfaces (e.g., fiber-optic alignment), communication channels (e.g., wireless protocols), functional integrations (e.g., software APIs), and any other form of association that achieves operational interaction.

Those skilled in the art will recognize that numerous modifications to the details of the above-described embodiments may be made without departing from the fundamental principles of the disclosed subject matter. Accordingly, the scope of the present disclosure shall be determined solely by the claims and their legal equivalents.

What is claimed is:

1. A method for presenting cardiac myocardial strain of a heart, wherein a left ventricle of the heart comprises a plurality of myocardial segments and a right ventricle of the heart comprises a plurality of myocardial segments, several of the myocardial segments are shared by both the left ventricle and the right ventricle, the method comprising:
acquiring first ultrasound image data of a cardiac imaging plane, the first ultrasound image data containing image data of the myocardial segments of the left ventricle and image data of the myocardial segments of the right ventricle;
processing the first ultrasound image data to obtain a motion parameter of the myocardial segments of the left ventricle and a motion parameter of the myocardial segments of the right ventricle;
displaying a composite bull's-eye plot of the left ventricle and the right ventricle, the composite bull's-eye plot comprising a left ventricular bull's-eye subplot and a right ventricular bull's-eye subplot; wherein:
(1) the left ventricular bull's-eye subplot comprises a plurality of left ventricular myocardial regions corresponding to the plurality of myocardial segments of the left ventricle, the motion parameter of the myocardial segments of the left ventricle is presented in its corresponding left ventricular myocardial regions; and,
the right ventricular bull's-eye subplot comprises at least one right ventricular myocardial region corresponding to the myocardial segments of the right ventricle excluding some or all of the shared myocardial segments, the motion parameter of said myocardial segments of the right ventricle is presented in its corresponding right ventricular myocardial region(s); or,
(2) the left ventricular bull's-eye subplot comprises at least one left ventricular myocardial region corresponding to the myocardial segments of the left ventricle excluding some or all of the shared myocardial segments, the motion parameter of said myocardial segments of the left ventricle is presented in its corresponding left ventricular myocardial region(s); and,
the right ventricular bull's-eye subplot comprises a plurality of right ventricular myocardial regions corresponding to the plurality of myocardial segments of the right ventricle, the motion parameter of the myocardial segments of the right ventricle is presented in its corresponding right ventricular myocardial regions.

2. The method according to claim 1, wherein:
a positional relationship between the left ventricular bull's-eye subplot and the right ventricular bull's-eye subplot in the composite bull's-eye plot is consistent with a positional relationship between the left ventricle and the right ventricle on a short-axis section; and,
the composite bull's-eye plot further comprises an interventricular septum region configured to present an interventricular septum, the interventricular septum region is adjacent to the left ventricular bull's-eye subplot and/or the right ventricular bull's-eye subplot.

3. The method according to claim 2, wherein: the right ventricular myocardial regions in the right ventricular bull's-eye subplot are sequentially arranged vertically along a curve;
the right ventricular bull's-eye subplot, the interventricular septum region, and the left ventricular bull's-eye subplot are arranged sequentially from left to right or from right to left; or,
the right ventricular bull's-eye subplot and the interventricular septum region are arranged vertically one above the other and both are adjacent to the left ventricular bull's-eye subplot.

4. The method according to claim 1, wherein:
a positional relationship between the left ventricular bull's-eye subplot and the right ventricular bull's-eye subplot in the composite bull's-eye plot is consistent with a positional relationship between the left ventricle and the right ventricle on a short-axis section; and the left ventricular bull's-eye subplot is adjacent to the right ventricular bull's-eye subplot.

5. The method according to claim 4 wherein:
the right ventricular myocardial regions in the right ventricular bull's-eye subplot are sequentially arranged vertically along a curve; or,
the right ventricular myocardial regions in the right ventricular bull's-eye subplot are arranged sequentially in a left-right orientation.

6. The method according to claim 2, wherein:
the right ventricular myocardial regions in the right ventricular bull's-eye subplot comprise one or more of:
a myocardial region corresponding to a top segment of a right ventricular free wall, a myocardial region corresponding to a mid segment of the right ventricular free wall, and a myocardial region corresponding to a bottom segment of the right ventricular free wall; and,
the left ventricular myocardial regions in the left ventricular bull's-eye subplot comprise: 16 myocardial regions corresponding respectively to 16 myocardial segments of the left ventricle, 17 myocardial regions corresponding respectively to 17 myocardial segments of the left ventricle, or 18 myocardial regions corresponding respectively to 18 myocardial segments of the left ventricle.

7. The method according to claim 1, further comprising:
acquiring second ultrasound image data of a/the cardiac imaging plane, the second ultrasound image data containing image data of a left atrium of the heart and image data of a right atrium of the heart;
processing the second ultrasound image data to obtain a motion parameter of the left atrium and a motion parameter of the right atrium; and,
displaying an atrial model diagram, the atrial model diagram being configured to display the motion parameter of the left atrium and the motion parameter of the right atrium.

8. The method according to claim 7, wherein:
the composite bull's-eye plot and the atrial model diagram are displayed in a same display interface; or,
displaying an atrial model diagram comprises: receiving a first switching instruction; and
switching from displaying the composite bull's-eye plot to displaying the atrial model diagram in response to the first switching instruction.

9. The method according to claim 7, wherein the atrial model diagram comprises: a left atrial region corresponding to the left atrium, a right atrial region corresponding to the right atrium, and an aortic region corresponding to an aorta of the heart; wherein the left atrial region displays the motion parameter of the left atrium, and the right atrial region displays the motion parameter of the right atrium.

10. The method according to claim 9, wherein the atrial model diagram further comprises: a right ventricular outflow tract region corresponding to a right ventricular outflow tract of the heart, and a pulmonary artery region corresponding to a pulmonary artery of the heart; and,
a positional relationship among the left atrial region, the right atrial region, the aortic region, the right ventricular outflow tract region and the pulmonary artery region in the atrial model diagram are consistent with positions of corresponding left atrium, right atrium, aorta, right ventricular outflow tract and pulmonary artery on a short-axis section.

11. The method according to claim 9, wherein the atrial model diagram further comprises: a right ventricular outflow tract region corresponding to a right ventricular outflow tract of the heart, and a pulmonary artery region corresponding to a pulmonary artery of the heart;
the aortic region is located centrally; and the left atrial region, the right atrial region, the right ventricular outflow tract region and the pulmonary artery region are sequentially arranged circumferentially around the aortic region.

12. The method according to claim 1, wherein processing the first ultrasound image data to obtain a motion parameter of the myocardial segments of the left ventricle and a motion parameter of the myocardial segments of the right ventricle, comprises:
determining a contour of the left ventricle and a contour of the right ventricle in the first ultrasound image data; and,
obtaining the motion parameter of the myocardial segments of the left ventricle and the motion parameter of the myocardial segments of the right ventricle, based on the contour of the left ventricle and the contour of the right ventricle in the first ultrasound image data.

13. The method according to claim 1, further comprising:
acquiring third ultrasound image data of a/the cardiac imaging plane, the third ultrasound image data containing image data of the myocardial segments of the left ventricle and image data of the left atrium;
processing the third ultrasound image data to obtain the motion parameter of the myocardial segments of the left ventricle and the motion parameter of the left atrium; and,
displaying a myocardial segment model diagram, wherein the myocardial segment model diagram comprises: a plurality of left ventricular myocardial regions corresponding to the plurality of myocardial segments of the left ventricle, and a left atrial region corresponding to the left atrium; the motion parameter of the myocardial segments of the left ventricle is presented in its corresponding left ventricular myocardial regions; and the motion parameter of the left atrium is presented in the left atrial region.

14. A method for presenting cardiac myocardial strain of a heart, comprising:
acquiring second ultrasound image data of a cardiac imaging plane, the second ultrasound image data containing image data of a left atrium of the heart and image data of a right atrium of the heart;
processing the second ultrasound image data to obtain a motion parameter of the left atrium and a motion parameter of the right atrium; and
displaying an atrial model diagram, the atrial model diagram being configured to display the motion parameter of the left atrium and the motion parameter of the right atrium, wherein the atrial model diagram comprises: a left atrial region corresponding to the left atrium, a right atrial region corresponding to the right atrium, and an aortic region corresponding to an aorta of the heart; the left atrial region displays the motion parameter of the left atrium; and the right atrial region displays the motion parameter of the right atrium.

15. The method according to claim 14, wherein the atrial model diagram further comprises: a right ventricular outflow tract region corresponding to a right ventricular outflow tract of the heart, and a pulmonary artery region corresponding to a pulmonary artery of the heart; and
a positional relationship among the left atrial region, the right atrial region, the aortic region, the right ventricular outflow tract region and the pulmonary artery region in the atrial model diagram are consistent with positions of corresponding left atrium, right atrium, aorta, right ventricular outflow tract, and pulmonary artery on a short-axis section.

16. The method according to claim 14, wherein the atrial model diagram further comprises: a right ventricular outflow tract region corresponding to a right ventricular outflow tract of the heart, and a pulmonary artery region corresponding to a pulmonary artery of the heart;
the aortic region is located centrally; and the left atrial region, the right atrial region, the right ventricular outflow tract region and the pulmonary artery region are sequentially arranged circumferentially around the aortic region.

17. The method according to claim 14, further comprising:
acquiring third ultrasound image data of a/the cardiac imaging plane, the third ultrasound image data containing image data of a plurality of myocardial segments of the left ventricle and image data of the left atrium;
processing the third ultrasound image data to obtain the motion parameter of the myocardial segments of the left ventricle and the motion parameter of the left atrium; and,
displaying a myocardial segment model diagram, wherein the myocardial segment model diagram comprises: a plurality of left ventricular myocardial regions corresponding to the plurality of myocardial segments of the left ventricle, and a left atrial region corresponding to the left atrium; the motion parameter of the myocardial segments of the left ventricle is presented in its corresponding left ventricular myocardial regions; and the motion parameter of the left atrium is presented in the left atrial region.

18. A method for presenting cardiac myocardial strain of a heart, wherein a left ventricle of the heart comprises a plurality of myocardial segments; the method comprising:
acquiring third ultrasound image data of a cardiac imaging plane, the third ultrasound image data containing image data of at least one of the myocardial segments of the left ventricle and image data of a left atrium of the heart;
processing the third ultrasound image data to obtain a motion parameter of the at least one of the myocardial segments of the left ventricle and a motion parameter of the left atrium; and
displaying a myocardial segment model diagram; wherein the myocardial segment model diagram comprises: a plurality of left ventricular myocardial regions corresponding to the plurality of myocardial segments of the left ventricle and a left atrial region corresponding to the left atrium, the motion parameter of the at least one of the myocardial segments of the left ventricle is presented in its corresponding left ventricular myocardial region(s), and the motion parameter of the left atrium is presented in the left atrial region.

19. The method according to claim 18, wherein:
the myocardial segment model diagram further comprises an aortic region corresponding to an aorta of the heart;
in the myocardial segment model diagram, the left ventricular myocardial regions are arranged to form a contour of the left ventricle on a cardiac imaging plane;
a positional relationship among the plurality of left ventricular myocardial regions, the left atrial region, and the aortic region in the myocardial segment model diagram are consistent with a positional relationship among corresponding left ventricle, left atrium, and aorta on a cardiac imaging plane.

20. The method according to claim 1, wherein the motion parameter comprises a strain, a strain rate, a velocity, a displacement, or a contrast agent time-to-peak.
